SEQUENCE LISTING (1 sequence)

SEQ ID NO: 1 - protein, 42 aa, Homo sapiens [sequence omitted]

(19) United States
(12) Patent Application Publication
(10) Pub. No.: US 2023/0218629 A1
(43) Pub. Date: Jul. 13, 2023

(54) THERAPEUTIC COMPOSITIONS AND METHODS FOR PREVENTION AND TREATMENT OF DIASTOLIC DYSFUNCTION

(71) Applicant: AMBETEX PTY LTD, Geelong West (AU)

(72) Inventor: Sean MCGEE, Geelong West (AU)

(73) Assignee: AMBETEX PTY LTD, Geelong West (AU)

(21) Appl. No.: 17/784,594

(22) PCT Filed: Dec. 9, 2020
(86) PCT No.: PCT/AU2020/051353

§ 371 Date: Jun. 10, 2022

(30) Foreign Application Priority Data
Dec. 11, 2019 (AU) 2019904683

(51) Int. Cl. A61K 31/549; A61P 9/04

(52) U.S. Cl. CPC A61K 31/549; A61P 9/04

(57) ABSTRACT

Methods for preventing or treating diastolic dysfunction in an individual comprising administering to an individual in need of said prevention or treatment a therapeutically effective amount of a (beta) β-secretase inhibitor, compositions comprising a (beta) β-secretase inhibitor for use in treatment of diastolic dysfunction and pharmaceutical compositions comprising a (beta) β-secretase inhibitor.

### FIELD OF THE INVENTION

The invention relates to diastolic dysfunction and related conditions and to β secretase inhibitors and therapeutic uses of same.

### BACKGROUND OF THE INVENTION

Diastole is the part of the cardiac cycle that includes the isovolumetric relaxation phase and the filling phases and has passive and active components. The filling of the left ventricle (LV) is divided into rapid filling during early diastole, diastasis, and a rapid filling phase late in diastole that corresponds with atrial contraction.

LV relaxation, an essential characteristic of normal diastole, is an energy-dependent process. In particular, adenosine triphosphate (ATP) is required to pump free myoplasmic calcium back into the sarcoplasmic reticulum, to extrude the calcium ions which enter the cell during the plateau phase of the action potential, and to extrude sodium that has been exchanged for calcium via sodium/potassium ATPase and an ATP-dependent calcium pump. Thus, when ATP production is limited, for example where there has been an impairment in the cardiac uptake of glucose, and/or impairments in mitochondrial metabolism, this may result in a slower rate of isovolumic relaxation and reduced distensibility of the LV.

Left ventricular diastolic dysfunction (LVDD) is a preclinical condition defined as the inability of the LV to fill an adequate end diastolic volume (preload volume) at an acceptable pressure. LVDD is generally a consequence of abnormalities during diastole. The aforementioned impaired LV relaxation, high filling pressure, and increased LV operating stiffness are underlying mechanisms in LVDD. Cardiac impairments that represent LVDD include reduced E:A ratio and increased deceleration time. These impairments can lead to concentric hypertrophy and associated cardiomyopathy, and heart failure.

Epidemiological evidence suggests there is a latent phase in which diastolic dysfunction is present and progresses in severity before the symptoms of heart failure arise. Asymptomatic mild LVDD is found in 21%, and moderate or severe diastolic dysfunction is present in 7% of the population.

In early diastolic dysfunction, elevated LV stiffness is associated with diastolic filling abnormalities and normal exercise tolerance. Asymptomatic diastolic dysfunction may be present for significant periods before it develops into a symptomatic clinical event. When the disease progresses, pulmonary pressures increase abnormally during exercise, producing reduced exercise tolerance. When filling pressures increase further, clinical signs of heart failure appear. In a significant number of cases of diastolic heart failure, patients have atrial fibrillation at the time of diagnosis, suggesting an association and a possible common pathogenesis. With atrial fibrillation, diastolic dysfunction may rapidly lead to overt diastolic heart failure.

The asymptomatic phase of diastolic dysfunction represents a potential time to intervene to prevent symptomatic heart failure. Suggesting the success of possible interventions, a mortality benefit has been observed in those whose diastolic dysfunction improved compared with those whose diastolic dysfunction remained the same or worsened.

Patients with LVDD are generally older, more often female, and have a high prevalence of CVD and other morbid conditions, such as obesity, metabolic syndrome, diabetes mellitus type 2, salt-sensitive hypertension, atrial fibrillation, COPD, anemia, and/or renal dysfunction.

LVDD may lead to heart failure with preserved ejection fraction (HFPeF). In HFPeF, normal ejection fraction is observed, but only at the expense of increased LV filling pressure. HFPeF is sometimes referred to as ‘diastolic heart failure’ or ‘backward heart failure’.

LVDD is an important precursor to many different cardiovascular diseases. It represents the dominant mechanism (⅔ of patients) in the development of HFPeF. HFPeF shows a rising prevalence in the older population. By 2020, more than 8% of people over 65 are estimated to have HFPEF and is associated with a poor prognosis.

To date, there are no specific treatments for diastolic dysfunction to selectively enhance myocardial relaxation. Moreover, no drug has been developed to improve long-term outcomes for diastolic heart failure.

WO2008/156828 discusses compounds that bind to aspartic proteases to inhibit their activity and the use of the compounds in the treatment or amelioration of diseases associated with aspartic protease activity.

WO2008/156816 discusses aspartic protease inhibitors and methods of antagonizing one or more aspartic proteases in a subject and methods for treating an aspartic protease mediated disorder.

US2012/0238548 discusses 1,4-oxazepines having BACE1 and/or BACE2 inhibitory activity and use of same for therapy or prophylaxis of Alzheimer's disease and type 2 diabetes.

WO2016/012384 discusses compounds having BACE1 inhibitor activity and use of same for therapy and prophylaxis of Alzheimer's disease.

WO2016/118404 discusses iminothiazine dioxides and use for treatment of Alzheimer's disease.

Tublin J. M et al., Circulation Research January 2019 Vol. 124 No. 1 discusses an overview of cardiovascular links to Alzheimer's disease.

Stamatelopoulos K et al. Rev Esp Cardio., 2017, Vol. 70, No. 11 discusses that circulating amyloid-beta (1-40) predicts clinical outcomes in patients with heart failure.

Schaich C et al. 2019 vol 7, no. 2:129-131 discusses a report of abnormal cardiac function in AD patients lacking symptomatic cardiovascular disease.

Troncone L et. al. J America College of Cardiol 2016 Vol. 68, No. 22 discusses if amyloid beta (Aβ) protein aggregates are present in the hearts of patients with a primary diagnosis of AD, affecting myocardial function.

There is a need for methods and compositions for providing improvements in the treatment or prevention of diastolic dysfunction.

### SUMMARY OF THE INVENTION

The invention relates to methods of treating, preventing, or ameliorating diastolic dysfunction or conditions associated with, or arising from same, and to pharmaceutical compositions and kits comprising β secretase inhibitors in an individual for treating or preventing diastolic dysfunction or conditions associated with, or arising from same.

The invention provides a method for preventing or treating diastolic dysfunction or condition associated with same in an individual comprising providing a therapeutically effective amount of a β secretase inhibitor in an individual.

The invention further provides a composition comprising a therapeutically effective amount of a β secretase inhibitor for use in preventing or treating diastolic dysfunction or condition associated with same in an individual.

The invention further provides a use of a composition comprising a β secretase inhibitor in the manufacture of a medicament for preventing or treating diastolic dysfunction or condition associated with same.

The invention further provides a method for preventing or treating diastolic dysfunction or condition associated with same in an individual comprising:
assessing or having assessed, a sample, preferably a plasma sample obtained from an individual for whom diastolic dysfunction is to be prevented or treated to determine the amount of Aβ42 in the sample; and
where the individual has an amount of Aβ42 that is greater than that observed in a control describing the amount of Aβ42 in an individual who does not develop, or does not have diastolic dysfunction;
   providing a β secretase inhibitor in the individual;
      thereby preventing or treating diastolic dysfunction or condition associated with same in the individual.

The invention further provides a kit comprising:
a β secretase inhibitor or pharmaceutical composition comprising same;
written instructions for use of the kit in an enumerated embodiment described below.

Various (enumerated) embodiments of the present invention are described herein. It will be recognized that features specified in each embodiment may be combined with other specified features to provide further embodiments of the present disclosure.

Embodiment 1: A method for preventing or treating diastolic dysfunction in an individual, preferably an obese, or pre-diabetic, or diabetic or elderly individual, more preferably an obese individual, or an individual having an elevated amount of Aβ42, more preferably an elevated plasma amount of Aβ42 comprising administering a therapeutically effective amount of a β secretase inhibitor to the individual, preferably wherein the β secretase inhibitor is selected from Table 3, more preferably wherein the β secretase inhibitor is compound 130 herein (verubecestat).

Embodiment 2: A method for preventing or treating heart failure, more preferably HFpEF in an individual, preferably an obese, or pre-diabetic, or diabetic or elderly individual, more preferably an obese individual, or an individual having an elevated amount of Aβ42, more preferably an elevated plasma amount of Aβ42 comprising administering a therapeutically effective amount of a β secretase inhibitor to the individual, preferably wherein the β secretase inhibitor is selected from Table 3, more preferably wherein the β secretase inhibitor is compound 130 herein (verubecestat).

Embodiment 3: A method for preventing or treating concentric hypertrophy in an individual, preferably an obese, or pre-diabetic, or diabetic or elderly individual, more preferably an obese individual, or an individual having an elevated amount of Aβ42, more preferably an elevated plasma amount of Aβ42 comprising administering a therapeutically effective amount of a β secretase inhibitor to the individual, preferably wherein the β secretase inhibitor is selected from Table 3, more preferably wherein the β secretase inhibitor is compound 130 herein (verubecestat).

Embodiment 4: A method for preserving or decreasing left ventricle deceleration time in an individual, preferably in an obese, or pre-diabetic, or diabetic or elderly individual, more preferably an obese individual, or an individual having an elevated amount of Aβ42, more preferably an elevated plasma amount of Aβ42 comprising administering a therapeutically effective amount of a β secretase inhibitor to the individual, preferably wherein the β secretase inhibitor is selected from Table 3, more preferably wherein the β secretase inhibitor is compound 130 herein (verubecestat).

Embodiment 5: A method for preserving or preventing intra-ventricular septal thickening in an individual, preferably in an obese, or pre-diabetic, or diabetic or elderly individual, more preferably an obese individual, or an individual having an elevated amount of Aβ42, more preferably an elevated plasma amount of Aβ42 comprising administering a therapeutically effective amount of a β secretase inhibitor to the individual, preferably wherein the β secretase inhibitor is selected from Table 3, more preferably wherein the β secretase inhibitor is compound 130 herein (verubecestat).

Embodiment 6: A method for preserving or preventing an increase in left ventricular mass in an individual, preferably in an obese, or pre-diabetic, or diabetic or elderly individual, more preferably an obese individual, or an individual having an elevated amount of Aβ42, more preferably an elevated plasma amount of Aβ42 comprising administering a therapeutically effective amount of a β secretase inhibitor to the individual, preferably wherein the β secretase inhibitor is selected from Table 3, more preferably wherein the β secretase inhibitor is compound 130 herein (verubecestat).

Embodiment 7: A method for preventing or treating cardiomyopathy, more preferably diabetic cardiomyopathy, or hypertrophic cardiomyopathy, or ischemic cardiomyopathy, or hypertensive cardiomyopathy in an individual, preferably an obese, or pre-diabetic, or diabetic or elderly individual, more preferably an obese individual, or an individual having an elevated amount of Aβ42, more preferably an elevated plasma amount of Aβ42 comprising administering a therapeutically effective amount of a β secretase inhibitor to the individual, preferably wherein the β secretase inhibitor is selected from Table 3, more preferably wherein the β secretase inhibitor is compound 130 herein (verubecestat).

Embodiment 8: A method for preventing the reduction of cardiac glucose uptake, or for preventing the accumulation cardiac tri-acyl glycerol in an individual, preferably an obese, or pre-diabetic, or diabetic or elderly individual, more preferably an obese individual, or an individual having an elevated amount of Aβ42, more preferably an elevated plasma amount of Aβ42 comprising administering a therapeutically effective amount of a β secretase inhibitor, preferably wherein the β secretase inhibitor is selected from Table 3, more preferably wherein the β secretase inhibitor is compound 130 herein (verubecestat).

Embodiment 9: A method for preventing or treating obesity-associated cardiomyopathy in an individual, more preferably in an individual having an elevated amount of Aβ42, more preferably an elevated amount of plasma Aβ42 comprising administering a therapeutically effective amount of a β secretase inhibitor, preferably wherein the β secretase inhibitor is selected from Table 3, more preferably wherein the β secretase inhibitor is compound 130 herein (verubecestat).

Embodiment 10: A composition for use in preventing or treating diastolic dysfunction in an individual, preferably an obese, or pre-diabetic, or diabetic or elderly individual, more preferably an obese individual, or an individual having an elevated amount of Aβ42, more preferably an elevated plasma amount of Aβ42 comprising a therapeutically effective amount of a β secretase inhibitor, preferably wherein the R secretase inhibitor is selected from Table 3, more preferably wherein the β secretase inhibitor is compound 130 herein (verubecestat).

Embodiment 11: A composition for use in preventing or treating heart failure, more preferably HFpEF in an individual, preferably an obese, or pre-diabetic, or diabetic or elderly individual, or an individual having an elevated amount of Aβ42, more preferably an elevated plasma amount of Aβ42 more preferably an obese individual, comprising a therapeutically effective amount of a β secretase inhibitor, preferably wherein the β secretase inhibitor is selected from Table 3, more preferably wherein the β secretase inhibitor is compound 130 herein (verubecestat).

Embodiment 12: A composition for use in preventing or treating concentric hypertrophy in an individual, preferably an obese, or pre-diabetic, or diabetic or elderly individual, more preferably an obese individual, or an individual having an elevated amount of Aβ42, more preferably an elevated plasma amount of Aβ42 comprising a therapeutically effective amount of a β secretase inhibitor, preferably wherein the β secretase inhibitor is selected from Table 3, more preferably wherein the β secretase inhibitor is compound 130 herein (verubecestat).

Embodiment 13: A composition for use in preserving or decreasing left ventricle deceleration time in an individual, preferably an obese, or pre-diabetic, or diabetic or elderly individual, more preferably an obese individual, or an individual having an elevated amount of Aβ42, more preferably an elevated plasma amount of Aβ42 comprising a therapeutically effective amount of a β secretase inhibitor, preferably wherein the β secretase inhibitor is selected from Table 3, more preferably wherein the β secretase inhibitor is compound 130 herein (verubecestat).

Embodiment 14: A composition for use in preserving or preventing intra-ventricular septal thickening in an individual preferably an obese, or pre-diabetic, or diabetic or elderly individual, more preferably an obese individual, or an individual having an elevated amount of Aβ42, more preferably an elevated plasma amount of Aβ42 comprising a therapeutically effective amount of a β secretase inhibitor, preferably wherein the β secretase inhibitor is selected from Table 3, more preferably wherein the β secretase inhibitor is compound 130 herein (verubecestat).

Embodiment 15: A composition for use in preserving or preventing an increase in left ventricular mass in an individual preferably an obese, or pre-diabetic, or diabetic or elderly individual, more preferably an obese individual, or an individual having an elevated amount of Aβ42, more preferably an elevated plasma amount of Aβ42 comprising a therapeutically effective amount of a β secretase inhibitor, preferably wherein the β secretase inhibitor is selected from Table 3, more preferably wherein the β secretase inhibitor is compound 130 herein (verubecestat).

Embodiment 16: A composition for use in preventing or treating cardiomyopathy, more preferably diabetic cardiomyopathy, or hypertrophic cardiomyopathy, or ischemic cardiomyopathy, or hypertensive cardiomyopathy in an individual, preferably an obese, or pre-diabetic, or diabetic or elderly individual, more preferably an obese individual, or an individual having an elevated amount of Aβ42, more preferably an elevated plasma amount of Aβ42 comprising a therapeutically effective amount of a β secretase inhibitor, preferably wherein the β secretase inhibitor is selected from Table 3, more preferably wherein the β secretase inhibitor is compound 130 herein (verubecestat).

Embodiment 17: A composition for use in preventing the reduction of cardiac glucose uptake, or for preventing the accumulation of cardiac tri-acyl glycerol in an individual, preferably an obese, or pre-diabetic, or diabetic or elderly individual, more preferably an obese individual, or an individual having an elevated amount of Aβ42, more preferably an elevated plasma amount of Aβ42 comprising a therapeutically effective amount of a β secretase inhibitor, preferably wherein the β secretase inhibitor is selected from Table 3, more preferably wherein the β secretase inhibitor is compound 130 herein (verubecestat).

Embodiment 18: A composition for use in preventing or treating obesity-associated cardiomyopathy in an individual, more preferably in an individual having an elevated amount of Aβ42, more preferably an elevated amount of plasma Aβ42 comprising a therapeutically effective amount of a 5 secretase inhibitor, preferably wherein the β secretase inhibitor is selected from Table 3, more preferably wherein the β secretase inhibitor is compound 130 herein (verubecestat).

### DETAILED DESCRIPTION OF THE INVENTION

The isovolumetric relaxation phase is an essential phase of normal diastole. It is energy dependent, and aberrations of the relaxation phase, as observed in LVDD and related clinical manifestations such as concentric hypertrophy and later heart failure, occur where there is an impairment in availability of ATP, for example as occurring where there is reduced cardiac glucose uptake.

It has been found herein that chronic exposure to Aβ42 results in impairments in cardiac metabolism, including reduction in cardiac glucose uptake, accumulation in cardiac TAG and impairment in cardiac function including concentric hypertrophy, and that these outcomes are minimised by minimising the exposure of cardiomyocytes to Aβ42 particularly those having a high fat content diet and/or whom are overweight or obese.

Without wanting to be bound by hypothesis it is believed that chronic exposure to Aβ42 causes or otherwise results in cardiomyocyte inflammation leading to impaired cardiac metabolism, reducing their glucose uptake, shunting of glucose into TAG and TAG accumulation, and that minimisation of exposure of cardiomyocytes to Aβ42 reduces these pathological outcomes.

Further, β secretase inhibitors are utilised herein to minimise the production of Aβ42, particularly Aβ42 production by adipocytes, in individuals in whom the prevention or treatment of LVDD is required.

### 1. Definitions

For purposes of interpreting this specification, the following definitions will apply and whenever appropriate, terms used in the singular will also include the plural and vice versa.

As used herein, the term “about” in relation to a numerical value x means+/−10%, unless the context dictates otherwise.

As used herein, the term “Amyloid beta” (Aβ or Abeta) denotes peptides of 36-43 amino acids, preferably Aβ42 that are crucially involved in Alzheimer's disease as the main component of the amyloid plaques found in the brains of Alzheimer patients. The peptides derive from the amyloid precursor protein (APP), which is cleaved by beta secretase and gamma secretase to yield Aβ. Aβ molecules can aggregate to form flexible soluble oligomers which may exist in several forms.

As used herein, the term “β secretase” or “beta secretase” or “betasite APP-cleaving enzyme” or “BACE-1” generally refers to a type-1 membrane-anchored aspartyl protease responsible for the first step of the proteolysis of APP. BACE-1 cleaves APP in the luminal surface of the plasma membrane and releases the soluble ectodomain of APP (sAPPA), leaving C99 (Aβ plus AICD (sometimes also referred to as “beta-CTF”), the latter which may then be subject to several cleavage events by gamma secretase to produce peptides of different lengths from 38 to 43 amino acids, including Aβ42. The activity of β-cleavage is considered to determine the total amount of Aβ production, and the efficiency of the successive γ-cleavage impacts the production ratio of toxic Aβ42 to total Aβ. BACE-1 is characterized by a large catalytic domain which is marked by the centrally located catalytic aspartates Asp32 and Asp228. Free BACE-1 features a flap-open conformation that is energetically stable due to the multiple hydrogen bonds in the flap region of the enzyme. When a substrate is bound, BACE-1 assumes a flap-closed or a flap-open conformation, depending on the characteristics of the substrate. The catalytic domain of BACE-1 contain eight pockets defined by the following amino acid residues:

S1: Leu30, Asp32, Tyr71, Leu119, Gln73, Phe108

S1′: Val31, Tyr71, Thr72, Asp228

S2: Asn233, Arg235, Ser325

S2′: Ser35, Val69, Pro70, Tyr198

S3: Leu133, Ile110, Ser229

S3′: Arg128, Tyr198

S4: Gln73, Thr232, Arg307

S4′: Glu125, Ile126, Tyr197, Tyr198

A enzyme “inhibitor” as used herein generally refers to a molecule that binds to an enzyme (for example β secretase) and thereby decreases its activity. The binding of the inhibitor hinders the enzyme from catalyzing a reaction. The binding of an inhibitory drug can either be irreversible or reversible. Irreversible inhibitors covalently bond with amino acid residues that are needed for the enzymatic activity, while reversible inhibitors bind non-covalently to either the enzyme itself, or the enzyme/substrate complex, through hydrogen bonds, ionic bonds or hydrophobic interactions. There are four different kinds of reversible enzyme inhibitors:
competitive inhibitors: the inhibitor has affinity for the active site of an enzyme where the substrate also binds. This leads the substrate and the inhibitor to compete for access to the enzyme's active site. Competitive inhibitors often mimic the structure of the natural substrates. Conversely, sufficiently high concentrations of the natural substrate, can out-compete the inhibitor and reduce its effects.
uncompetitive inhibition: the inhibitor binds to the enzyme/substrate complex, hindering the catalysis of the natural substrate.
mixed inhibitors: when the inhibitor binds to the enzyme, it affects the enzyme's binding to the substrate and vice versa. It is possible for these inhibitors to bind at the active site, but inhibition generally occurs from an allosteric effect where the inhibitor binds adjacent to the active site, changing the conformation of the enzyme. This results in reduced affinity of the substrate for the active site.
non-competitive inhibitors: binding of the inhibitor to the enzyme reduces enzyme activity, but does not affect the binding of a substrate to the active site. The concentration of the inhibitor determines the extent of inhibition.

As used herein, the term “β secretase inhibitor” generally refers to a compound that inhibits the cleavage of APP by a β secretase, thereby inhibiting the generation of the C99 (or beta-CTF) fragment referred to above, and thereby depriving gamma secretase of substrate for generation of the 38 to 43 amino acid peptides, including Aβ42 referred to above. β secretase inhibitors contemplated for use in the invention are described further herein.

As used herein, the term “diastolic dysfunction” generally refers to a condition characterised by the inability of the left ventricle to fill an adequate end diastolic volume at a physiologically normal or acceptable pressure.

As used herein, the term “E/A ratio” generally refers to the ratio of the E wave to the A wave. On echocardiography, the peak velocity of blood flow across the mitral valve during early diastolic filling corresponds to the E wave. Similarly, atrial contraction corresponds to the A wave. From these findings, “the E/A ratio” is calculated. Under normal conditions, E is greater than A and the E/A ratio is approximately 1.5. In early diastolic dysfunction, relaxation is impaired and, with vigorous atrial contraction, the E/A ratio decreases to less than 1.0. As the disease progresses, left ventricular compliance is reduced, which increases left atrial pressure and, in turn, increases early left ventricular filling despite impaired relaxation. This paradoxical normalization of the E/A ratio may be called “pseudonormalization”. In patients with severe diastolic dysfunction, left ventricular filling occurs primarily in early diastole, creating an E/A ratio greater than 2.0.

As used herein, “deceleration time” is the time taken from the maximum E point to baseline. In adults, it is normally less than 220 milliseconds.

As used herein, the term “concentric hypertrophy” generally refers to a form of cardiac hypertrophy associated with increased left ventricular wall thickness, or associated with an increase in LV mass without dilation of the LV, for example as measured by LVIDd. An increase in pressure, common in hypertension or resistance training, results in a concentric hypertrophic phenotype. Concentric hypertrophy differs from “eccentric hypertrophy”, the latter being characterised by dilatation of the left ventricular chamber and is observed in, or associated with valvular defects or endurance training. Eccentric hypertrophy may develop from concentric hypertrophy. An individual with diastolic dysfunction, in particular, an individual with early stage diastolic dysfunction may or may not have detectable concentric hypertrophy.

As used herein, the term “HFpEF” or “heart failure with preserved ejection fraction” generally refers to a form of heart failure characterised by normal ejection fraction (at or above about 50% of ventricle volume) dependent on increased LV pressure.

As used herein, “Cardiomyopathy” generally refers to a heterogeneous group of diseases of the myocardium associated with mechanical and/or electrical dysfunction, which usually (but not invariably) exhibit inappropriate ventricular hypertrophy or dilatation. Cardiomyopathy may be a primary cardiomyopathy, which is confined to the heart, preferably an acquired cardiomyopathy, more preferably an obesity-associated cardiomyopathy. An obesity-associated cardiomyopathy is defined myocardial disease in obese individuals that cannot be explained by diabetes mellitus, hypertension, coronary artery disease or other etiologies. The presentation of this disease can vary from asymptomatic left ventricular dysfunction to overt dilated cardiomyopathy and heart failure.

As used herein, the term “elderly individual” refers to an individual over 60 years of age, more preferably 65 or 70 or 75 years of age.

As used herein, the term “pharmaceutically acceptable” means a nontoxic material that does not interfere with the effectiveness of the biological activity of the active ingredient(s).

As used herein, the term “treat”, “treating” or “treatment” in connection to a disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, {e.g., stabilization of a discernible symptom), physiologically, {e.g., stabilization of a physical parameter), or both. The term “alleviating” or “alleviation”, for example in reference to a symptom of a condition, as used herein, refers to reducing at least one of the frequency and amplitude of a symptom of a condition in a patient. In one embodiment, the terms “method for the treatment” or “method for treating”, as used herein, refer to “method to treat”.

As used herein, the term “therapeutically effective amount” refers to an amount of the compound of the invention, e.g. β secretase inhibitor; which is sufficient to achieve the stated effect. Accordingly, a therapeutically effective amount of a β secretase inhibitor; will be an amount sufficient for the treatment or prevention of the condition mediated by or associated with Aβ42 plasma expression or production.

By “therapeutic regimen” is meant the pattern of treatment of an illness, e.g., the pattern of dosing used during the treatment of the disease or disorder.

As used herein, a subject is “in need of” a treatment if such subject would benefit biologically, medically or in quality of life from such treatment.

### 2. DETAILED DESCRIPTION OF THE FIGURES

FIG. 1—Chronic Aβ42 administration alters cardiac metabolism.

FIG. 2—Chronic Aβ42 administration alters cardiac function.

FIG. 3—Administration of anti-Aβ42 antibodies preserves diastolic function in development of obesity.

FIG. 4—Administration of anti-Aβ42 antibodies prevents concentric hypertrophy in development of obesity.

FIG. 5—Administration of anti-Aβ42 antibodies preserves diastolic function in established obesity.

FIG. 6—Chronic Aβ40 administration does not alter cardiac function.

FIG. 7—Administration of β secretase inhibitor preserves diastolic function in established obesity.

### 3. MODES OF CARRYING OUT THE INVENTION

### 3.1 Individuals

An individual to whom the methods of the invention are applied is mammalian, preferably a human being.

An individual may not have diastolic dysfunction at the time of treatment. Such an individual may be at risk for diastolic dysfunction i.e. may have one or more risk factors for diastolic dysfunction. For example, the individual may be pre diabetic or diabetic, overweight or obese, female, have Alzheimer's disease or other neural disease with Aβ involvement, or elderly. The individual may have an elevated amount of Aβ42, preferably an elevated amount of plasma Aβ42. The invention may be applied to such an individual to prevent the development of diastolic dysfunction, or to prevent diastolic dysfunction.

In another embodiment, an individual may have diastolic dysfunction at the time of treatment. Such an individual may be asymptomatic for diastolic dysfunction, or symptomatic for diastolic dysfunction. The invention may be applied to such an individual to treat or ameliorate or alleviate diastolic dysfunction.

In one embodiment, the individual to be administered a secretase inhibitor is obese and has an elevated amount of plasma Aβ42 and may or may not have diastolic dysfunction. Such an individual may have obesity associated cardiomyopathy, or may be at risk for same.

Stages of diastolic dysfunction have been classified according to various grading systems. For example, four basic echocardiographic patterns of diastolic dysfunction, (graded I to IV) according to the American Society of Echocardiography and the European Association of Cardiovascular Imagining are described:
Grade I diastolic dysfunction. On the mitral inflow Doppler echocardiogram, the E/A ratio is ≤0.8 and deceleration time is >200 ms, while the E/e′ ratio, a measure of the filling pressure, is within normal limits at <10. This pattern may develop normally with age in some patients, and many grade I patients will not have any clinical signs or symptoms of heart failure.
Grade II diastolic dysfunction is called “pseudonormal filling dynamics”, with the E/A ratio between 0.8 and 2.0, and a reduction in deceleration time to between 160 and 220 ms. This is considered moderate diastolic dysfunction and is associated with elevated left atrial filling pressures with an E/e′ ratio between 10 and 14. These patients more commonly have symptoms of heart failure, and many have left atrial enlargement due to the elevated pressures in the left heart.
Class III diastolic dysfunction patients will have an E/A ratio >2 and E/e′ ratio >14. They will demonstrate reversal of their diastolic abnormalities on echocardiogram when they perform the Valsalva maneuver. This is referred to as “reversible restrictive diastolic dysfunction”.
Class IV diastolic dysfunction patients will not demonstrate reversibility of their echocardiogram abnormalities, and are therefore said to suffer from “fixed restrictive diastolic dysfunction”.

Grade III and IV diastolic dysfunction are called “restrictive filling dynamics”. These are both severe forms of diastolic dysfunction, and patients tend to have advanced heart failure symptoms.

In one embodiment, an individual having Grade I diastolic dysfunction (as described above), preferably having an elevated plasma amount of Aβ42 is provided with a β secretase inhibitor to prevent the development of more severe diastolic dysfunction, or otherwise to preserve diastolic function.

In one embodiment, an individual having Grade II, III or IV diastolic dysfunction (as described above), preferably having an elevated plasma amount of Aβ42 is provided with a β secretase inhibitor to treat or reverse diastolic dysfunction, or to treat or reverse one or more symptoms or characters of diastolic dysfunction.

In one embodiment, an individual may have concentric hypertrophy.

An individual in need of treatment may have a normal left ventricle diameter and may have a normal cardiac weight.

An individual in need of treatment may have an increased LV deceleration time.

An individual in need of treatment may have a cardiomyopathy, especially an ischemic or hypertrophic cardiomyopathy.

An individual in need of treatment may have a systolic condition in addition to diastolic dysfunction.

An individual the subject of treatment may be symptomatic for heart failure and may be symptomatic for HFPpEF or may be asymptomatic for heart failure or HFpEF. Symptoms of heart failure generally include shortness of breath including exercise induced dyspnea, paroxysmal nocturnal dyspnea and orthopnea, exercise intolerance, fatigue, elevated jugular venous pressure, and edema. Patients with HFpEF poorly tolerate stress, particularly hemodynamic alterations of ventricular loading or increased diastolic pressures. Often there is a more dramatic elevation in systolic blood pressure in HFpEF.

An individual who is asymptomatic or symptomatic for heart failure may or may not be obese or overweight, diabetic or pre-diabetic, have Alzheimer's disease or other neural disease with Aβ involvement, or elderly.

### 3.2 Screening Individuals for LVDD

In a particularly preferred embodiment, an individual may be selected for treatment or prevention of LVDD, or screened for LVDD, or assessed for risk of developing LVDD by assessing or measuring the plasma amount of Aβ and optionally comparing with a normal control describing an amount of Aβ in plasma in an individual not having, or not at risk of having diastolic dysfunction, for example, an individual who is not overweight or obese, or not pre-diabetic or diabetic, or who does not have Alzheimer's disease or who is not elderly.

In one embodiment, a control may be an age matched control. Where the individual to be assessed is elderly, the control may describe an amount of Aβ42 in plasma that is consistent with that found in a normal individual having an age of about 20 to 40 years old.

In one embodiment a control describes the amount of Aβ42 in plasma from an individual having a body mass index in the normal range, from about 18.5 to 24.9 kg/m².

In one embodiment, a control describing the amount of Aβ42 in plasma may be may be derived from a single individual. In another embodiment, a control may be derived from a cohort of individuals.

It has been established in the Examples herein that diastolic dysfunction is induced by administration of an amount of about 0.04 mg/kg of Aβ42 peptide per day. Further, individuals on a high fat diet may develop a plasma amount of Aβ42 peptide of about 3 fold above controls. In one embodiment, an individual to be selected for treatment may have a plasma amount of Aβ42 peptide of about 10 to 100 pM, or about 1 to at least 10 fold the amount of Aβ42 peptide in a control.

A control may provide a reference point against which a determination regarding implementation of subsequent prophylaxis or therapy can be made. The determination may be made on the basis of the comparison between test sample obtained from the individual being assessed for prophylaxis or treatment and the control.

In certain embodiments, the control may be provided in the form of data that has been derived by another party, and/or prior to assessment of the subject for treatment. For example, the control may be derived from a commercial database or a publically available database.

In one embodiment the individual is selected for treatment or prevention of LVDD, or screened for LVDD, or assessed for risk of developing LVDD, where the individual has an amount of Aβ or fragment thereof, preferably Aβ42 that is greater than the amount of Aβ or fragment thereof, preferably Aβ42 in a normal control.

Methods for measurement of plasma amounts of Aβ or fragment thereof, such as Aβ42 are known in the art: [Kim et al., Sci. Adv. 2019; 5:eaav1388 17 Apr. 2019; Shie, F S et al., PLOSONE|DOI:10.1371/journal.pone.0134531 Aug. 5, 2015; Balakrishnan K et al. Journal of Alzheimer's Disease 8 (2005) 269-282; Luciano R et al., PEDIATRICS Volume 135, number 6, June 2015].

In certain embodiments, the samples to be tested are body fluids such as blood, serum, plasma, urine, tears, saliva, CSF and the like.

In certain embodiments, the sample from the individual may require processing prior to detection of the levels of Aβ42. For example, the sample may be centrifuged or diluted to a particular concentration or adjusted to a particular pH prior to testing. Conversely, it may be desirable to concentrate a sample that is too dilute, prior to testing.

In certain embodiments Aβ42 may be measured, or peptides or complexes that comprise Aβ42 may be measured.

In other embodiments, fragments of Aβ42 comprising the Aβ42 C-terminal sequences that distinguish Aβ42 from Aβ40 may be measured.

The above described methods may be combined with the following diagnostic procedures for detecting, assessing or measuring diastolic dysfunction or related heart failure such as HFPeF, or the following procedures may be used without assessment of plasma amount of Aβ42.

Two-dimensional echocardiography with Doppler flow measurements is commonly used to assess diastolic dysfunction. Exercise may be required to clearly demonstrate diastolic functional changes. During diastole, blood flows through the mitral valve when the LV relaxes, causing an early diastolic mitral velocity (E), and then additional blood is pumped through the valve when the left atrium contracts during late diastole (A). The E/A ratio can be altered in diastolic dysfunction.

Tissue Doppler imaging is an echocardiographic technique that measures the velocity of the mitral annulus. This velocity has been shown to be a sensitive marker of early myocardial dysfunction. With abnormal active relaxation, mitral annulus velocity during early diastole (E) is decreased while mitral annulus velocity during late diastole (A) is increased, resulting in a lowered E/A ratio. In animal models, tissue Doppler imaging has been validated as a reliable tool for the evaluation of diastolic dysfunction.

The E- and A-wave velocities are affected by blood volume, mitral valve anatomy, mitral valve function, and atrial fibrillation, making standard echocardiography less reliable. In these cases, tissue Doppler imaging is useful for measuring mitral annular motion (a measure of transmitral flow that is independent of the aforementioned factors). Cardiac catheterization remains the preferred method for diagnosing diastolic dysfunction. However, in day-to-day clinical practice, two-dimensional echocardiography with Doppler is the best noninvasive tool to confirm the diagnosis. Rarely, radionuclide angiography is used for patients in whom echocardiography is technically difficult.

LV inflow propagation velocity (VP) by color M-mode Doppler is another relatively preload-insensitive index of LV relaxation. It has been shown to correlate well with the time constant of isovolumic relaxation (T), both in animals and humans.

Recently, speckle tracking echocardiography (STE) has emerged as a promising technique for the evaluation of myocardial wall motion by strain analysis. By tracking the displacement of speckles during the cardiac cycle, STE allows semiautomated delineation of myocardial deformation.

Cardiac magnetic resonance (CMR) imaging is a newer technique for measuring diastolic dysfunction. Myocardial tagging allows the labeling of specific myocardial regions. Following these regions during diastole enables them to be analyzed in a manner similar to STE. In addition, the rapid diastolic untwisting motion followed by CMR tagging is directly related to isovolumic relaxation and can be used as an index of the rate and completeness of relaxation.

Biomarkers may also be assessed for diagnosis of LVDD. B-type natriuretic peptide (BNP) and TnI have been used as HF biomarkers and exhibit strong association with hospitalization. Nevertheless, they are nonspecific and not well correlated with diastolic dysfunction. Recently, it has been reported that cMyBP-C could be a new biomarker releases from damaged myofilaments. Additionally, elevated S-glutathionylated cMyBP-C level can be detected in the blood of patients with diastolic dysfunction. Hypertension and diabetes lead to cardiac oxidation and S-glutathionylation of cMyBP-C, a cardiac contractile protein, which leads to impaired relaxation, and modified cMyBP-C in the blood may represent a circulating biomarker for diastolic dysfunction.

### 3.3 β Secretase Inhibitors

β secretase inhibitors for use in the invention generally inhibits the cleavage of APP by a β secretase, thereby inhibiting the generation of the C99 (or beta-CTF). Without wanting to be bound by hypothesis, it is believed that the administration of a β secretase inhibitor minimises the amount of plasma Aβ, resulting in a minimisation of diastolic dysfunction, preferably through a minimisation of Aβ induced or associated cardiomyocyte inflammation and/or reduced cardiac glucose uptake.

Methods for assessment of β-secretase inhibitor activity, i.e. for measurement of soluble APPβ (sAPPβ) formed by BACE-1 cleavage of APP, and for measurement of Aβ40 and Aβ41 are known and described in for example, Villarreal S. et al. 2019 J. Alz Disease 59:1393-1413.

Inhibitors may bind to β-secretase through non-covalent interactions and therefore may be reversible inhibitors. Inhibition may depend on β-secretase having a greater affinity for the inhibitor than for APP, in which case inhibition is a form of competitive inhibition. Maximal inhibitor affinity may be created by increasing the number of noncovalent interactions between BACE1 and the inhibitor. Aspartic proteases like BACE1 have a catalytic domain containing a pair of active-site aspartic acid residues. Furthermore, BACE1 has an elongated substrate-binding site, the subsites, that can bind up to 11 amino acids of substrates. These substrates are processed with the aid of the two aspartic acid residues in the active site. Inhibitor specificity for BACE1 can be built by taking advantage of the collective interactions between a putative inhibitor and a part of the substrate-binding groove of BACE1. The 11 subsites have a broad amino acid preference, but many central ones (such as P1 and P1′) prefer hydrophobic side chains.

β secretase inhibitors may generally be classified as peptidomimetic-based inhibitors (for example, statine or norstatine-based inhibitors; hydroxyethylene-based inhibitors; hydroxyethyamine-based inhibitors; carbamine-based inhibitors; reduced amide-based inhibitors and peptide like macrocyclic-based inhibitors) or non peptide-based inhibitors (for example acyl-guanine-based inhibitors; 2-amino pyridine-based inhibitors; amino-imidazole-based inhibitors; amino/imino hydantoin-based inhibitors; aminothiazoline and aminooxazoline-based inhibitors; dihydroquinalzoline-based inhibitors; aminoquinoline-based inhibitors; pyrrolidine-based inhibitors; and macrocyclic non peptide-based inhibitors).

There now follows a discussion of β secretase inhibitors contemplated for use in the invention.

### 3.3.1 Peptidomimetic-Based Inhibitors

A β secretase inhibitor contemplated for use in the invention may be a peptidomimetic-based inhibitor. A peptidomimetic may mimic the sequence of a BACE-1 substrate, such as APP.

Peptidomimetics that are protease inhibitors may be developed by product screening for lead substrates by substrate-based methods and subsequent optimization. Such optimization includes the replacement of hydrolyzable peptide bonds by non-hydrolyzable isosteres. An isotere may bind more tightly to an aspartic protease than does a natural substrate bind to an aspartic protease. In free BACE1, a catalytic aspartic acid residue hydrogen bonds to a water molecule, holding it in place. When encountered with a substrate molecule, one of the aspartic acids assists nucleophilic attack of the water molecule at the carbonyl carbon while the other aspartic acid residue activates the carbonyl of the peptide bond, eventually leading to substrate cleavage. A peptidomimetic-based inhibitor may intervene in this process when the hydroxyl group of the transition state isotere displaces the water molecule, thus forming tight hydrogen bonds to the catalytic aspartic acid residues and precluding substrate cleavage.

Peptidomimetics for use in the invention inhibit the enzymatic action of β secretases especially in adipocytes and fat tissue. Therefore, it is not essential that a peptidomimetic for use in the invention should be capable of crossing the blood brain barrier (BBB). In one embodiment, a peptidomimetic for use as a β secretase inhibitor according to the invention does not cross the BBB or has limited capacity to cross the BBB.

Examples of peptidomimetic-based inhibitors contemplated for use as β secretase inhibitors according to the invention may be statine or norstatine-based inhibitors; hydroxyethylene-based inhibitors; hydroxyethyamine-based inhibitors; carbamine-based inhibitors; reduced amide-based inhibitors as shown below:

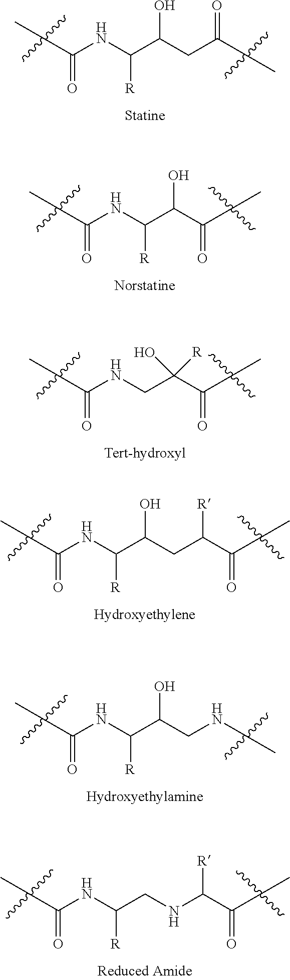

### 3.3.1.1 Statine or Norstatine-Based Inhibitors

In one embodiment a peptidomimetic-based inhibitor is a statine or norstatine-based inhibitor. Examples of these inhibitors are shown in Tables 1a. 1d and 1e.

TABLE 1a

| Compound # | Structure |
|---|---|
| 1a | 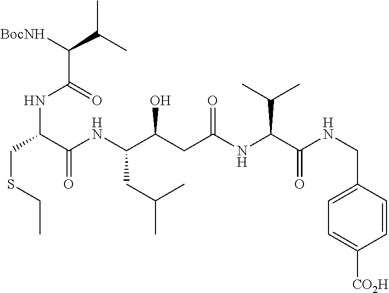 [SMILES: CCSC[C@H](NC(=O)[C@@H](NC(=O)OC(C)(C)C)C(C)C)C(=O)N[C@@H](CC(C)C)[C@@H](O)CC(=O)N[C@H](C(=O)NCc1ccc(C(=O)O)cc1)C(C)C] |
| 1b | 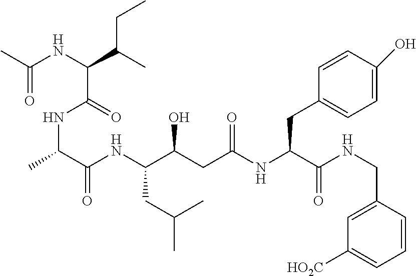 [SMILES: CCC(C)[C@H](NC(C)=O)C(=O)N[C@@H](C)C(=O)N[C@@H](CC(C)C)[C@@H](O)CC(=O)N[C@@H](Cc1ccc(O)cc1)C(=O)NCc1cccc(C(=O)O)c1] |
| 1c | 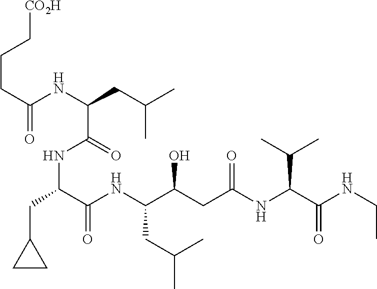 [SMILES: CCNC(=O)[C@@H](NC(=O)C[C@H](O)[C@H](CC(C)C)NC(=O)[C@H](CC1CC1)NC(=O)[C@H](CC(C)C)NC(=O)CCCC(=O)O)C(C)C] |
| 1d | 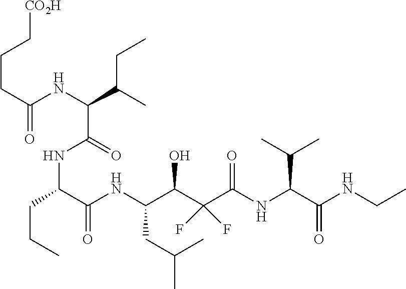 [SMILES: CCC[C@H](NC(=O)[C@@H](NC(=O)CCCC(=O)O)C(C)CC)C(=O)N[C@@H](CC(C)C)[C@@H](O)C(F)(F)C(=O)N[C@H](C(=O)NCC)C(C)C] |
| 1e | 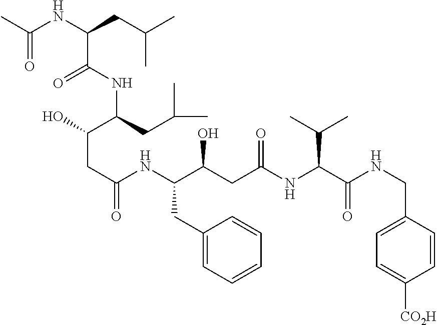 [SMILES: CC(=O)N[C@@H](CC(C)C)C(=O)N[C@@H](CC(C)C)[C@@H](O)CC(=O)N[C@@H](Cc1ccccc1)[C@@H](O)CC(=O)N[C@H](C(=O)NCc1ccc(C(=O)O)cc1)C(C)C] |
| 1f | 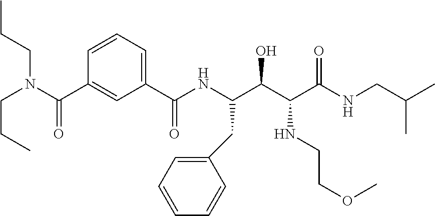 |
| 1g | 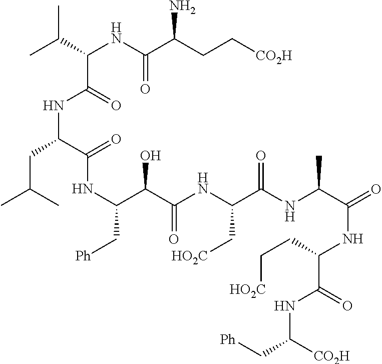 [SMILES: CC(C)C[C@H](NC(=O)[C@@H](NC(=O)[C@@H](N)CCC(=O)O)C(C)C)C(=O)N[C@@H](Cc1ccccc1)[C@@H](O)C(=O)N[C@@H](CC(=O)O)C(=O)N[C@@H](C)C(=O)N[C@@H](CCC(=O)O)C(=O)N[C@@H](Cc1ccccc1)C(=O)O] |
| 1h | 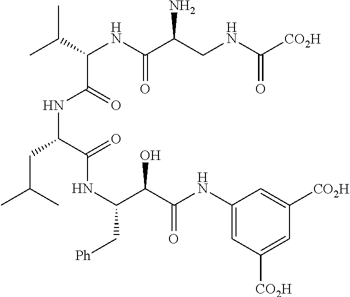 [SMILES: CC(C)C[C@H](NC(=O)[C@@H](NC(=O)[C@@H](N)CNC(=O)C(=O)O)C(C)C)C(=O)N[C@@H](Cc1ccccc1)[C@@H](O)C(=O)Nc1cc(C(=O)O)cc(C(=O)O)c1] |
| 1i | 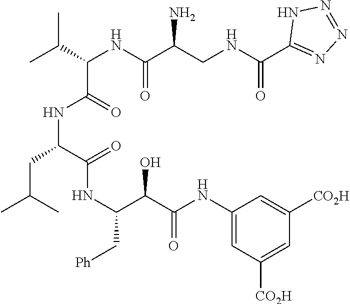 [SMILES: CC(C)C[C@H](NC(=O)[C@@H](NC(=O)[C@@H](N)CNC(=O)c1nnn[nH]1)C(C)C)C(=O)N[C@@H](Cc1ccccc1)[C@@H](O)C(=O)Nc1cc(C(=O)O)cc(C(=O)O)c1] |
| 1j | 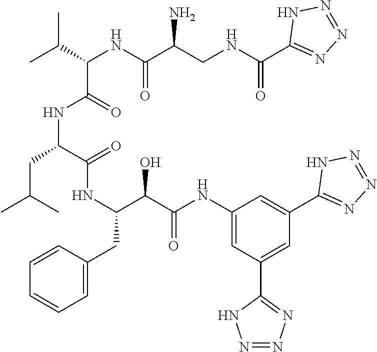 [SMILES: CC(C)C[C@H](NC(=O)[C@@H](NC(=O)[C@@H](N)CNC(=O)c1nnn[nH]1)C(C)C)C(=O)N[C@@H](Cc1ccccc1)[C@@H](O)C(=O)Nc1cc(-c2nnn[nH]2)cc(-c2nnn[nH]2)c1] |
| 1k | 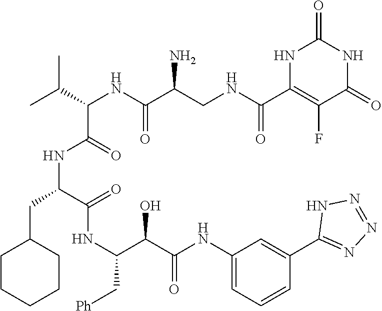 [SMILES: CC(C)[C@H](NC(=O)[C@@H](N)CNC(=O)c1[nH]c(=O)[nH]c(=O)c1F)C(=O)N[C@@H](CC1CCCCC1)C(=O)N[C@@H](Cc1ccccc1)[C@@H](O)C(=O)Nc1cccc(-c2nnn[nH]2)c1] |
| 1l | 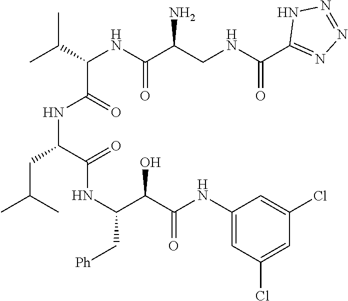 [SMILES: CC(C)C[C@H](NC(=O)[C@@H](NC(=O)[C@@H](N)CNC(=O)c1nnn[nH]1)C(C)C)C(=O)N[C@@H](Cc1ccccc1)[C@@H](O)C(=O)Nc1cc(Cl)cc(Cl)c1] |
| 1m | 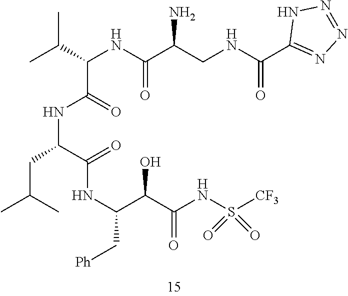 [SMILES: CC(C)C[C@H](NC(=O)[C@@H](NC(=O)[C@@H](N)CNC(=O)c1nnn[nH]1)C(C)C)C(=O)N[C@@H](Cc1ccccc1)[C@@H](O)C(=O)NS(=O)(=O)C(F)(F)F] |
| 1n | 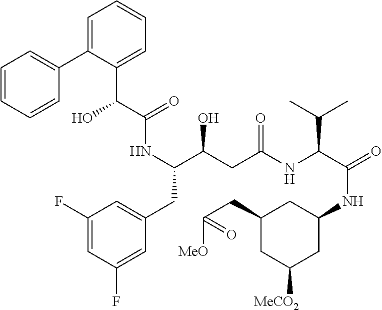 |
| 1o | 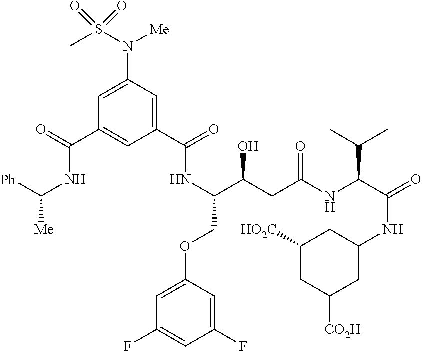 [SMILES: CC(C)[C@H](NC(=O)C[C@H](O)[C@H](COc1cc(F)cc(F)c1)NC(=O)c1cc(C(=O)N[C@H](C)c2ccccc2)cc(N(C)S(C)(=O)=O)c1)C(=O)NC1CC(C(=O)O)C[C@H](C(=O)O)C1] |

### 3.3.1.2 Tert-Hydroxyl-Based Inhibitors

In one embodiment a peptidomimetic-based inhibitor is a tert-hydroxyl-based inhibitor. Examples of these inhibitors are shown in Table 1b

TABLE 1b

| Compound # | Structure |
|---|---|
| 18p | 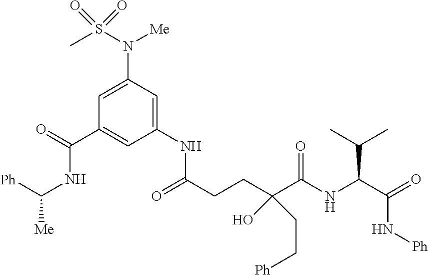 [SMILES: CC(C)[C@H](NC(=O)C(O)(CCC(=O)Nc1cc(C(=O)N[C@H](C)c2ccccc2)cc(N(C)S(C)(=O)=O)c1)CCc1ccccc1)C(=O)Nc1ccccc1] |
| 1q | 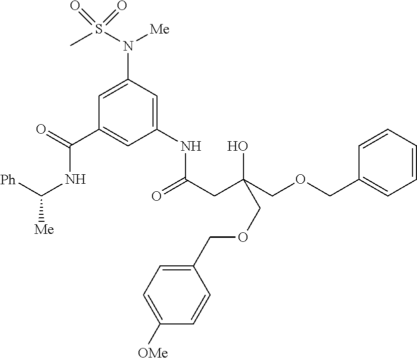 [SMILES: COc1ccc(COCC(O)(COCc2ccccc2)CC(=O)Nc2cc(C(=O)N[C@H](C)c3ccccc3)cc(N(C)S(C)(=O)=O)c2)cc1] |
| 1r | 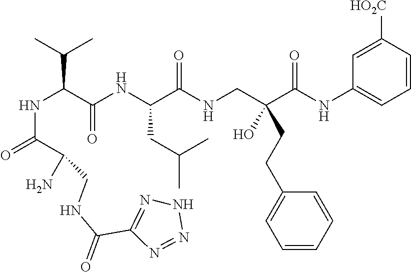 [SMILES: CC(C)C[C@H](NC(=O)[C@@H](NC(=O)[C@@H](N)CNC(=O)c1nn[nH]n1)C(C)C)C(=O)NC[C@](O)(CCc1ccccc1)C(=O)Nc1cccc(C(=O)O)c1] |

### 3.3.1.3 Hydroxyethylene-Based Inhibitors

In one embodiment a peptidomimetic-based inhibitor is a hydroxyethylene-based inhibitor. Examples of these inhibitors are shown in Tables 1c, 1d or 1e.

TABLE 1c

| Compound # | Structure |
|---|---|
| 1s | 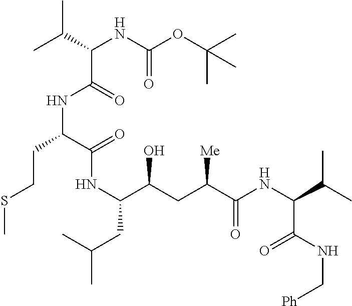 [SMILES: CSCC[C@H](NC(=O)[C@@H](NC(=O)OC(C)(C)C)C(C)C)C(=O)N[C@@H](CC(C)C)[C@@H](O)C[C@@H](C)C(=O)N[C@H](C(=O)NCc1ccccc1)C(C)C] |
| 1t | 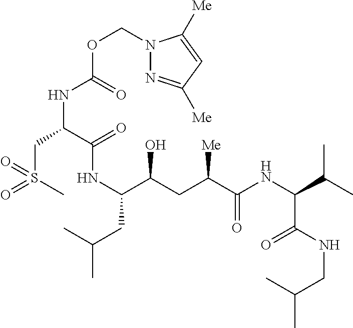 [SMILES: Cc1cc(C)n(COC(=O)N[C@@H](CS(C)(=O)=O)C(=O)N[C@@H](CC(C)C)[C@@H](O)C[C@@H](C)C(=O)N[C@H](C(=O)NCC(C)C)C(C)C)n1] |
| 1u | 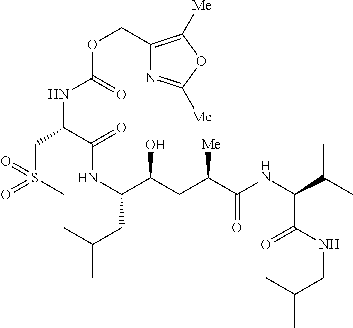 [SMILES: Cc1nc(COC(=O)N[C@@H](CS(C)(=O)=O)C(=O)N[C@@H](CC(C)C)[C@@H](O)C[C@@H](C)C(=O)N[C@H](C(=O)NCC(C)C)C(C)C)c(C)o1] |
| 1v | 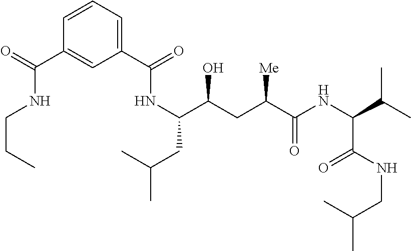 [SMILES: CCCNC(=O)c1cccc(C(=O)N[C@@H](CC(C)C)[C@@H](O)C[C@@H](C)C(=O)N[C@H](C(=O)NCC(C)C)C(C)C)c1] |
| 1w | 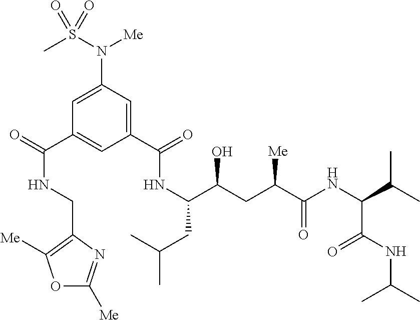 [SMILES: Cc1nc(CNC(=O)c2cc(C(=O)N[C@@H](CC(C)C)[C@@H](O)C[C@@H](C)C(=O)N[C@H](C(=O)NC(C)C)C(C)C)cc(N(C)S(C)(=O)=O)c2)c(C)o1] |
| 1y | 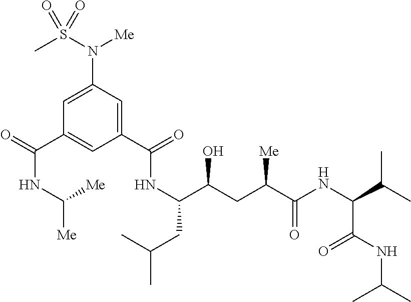 |
| 1z | 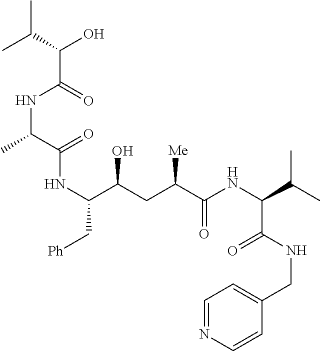 [SMILES: CC(C)[C@H](O)C(=O)N[C@@H](C)C(=O)N[C@@H](Cc1ccccc1)[C@@H](O)C[C@@H](C)C(=O)N[C@H](C(=O)NCc1ccncc1)C(C)C] |
| 1aa | 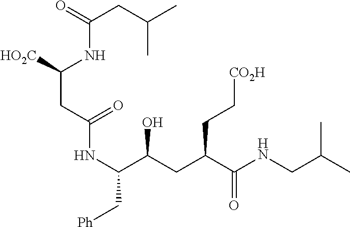 [SMILES: CC(C)CNC(=O)[C@H](CCC(=O)O)C[C@H](O)[C@H](Cc1ccccc1)NC(=O)C[C@H](NC(=O)CC(C)C)C(=O)O] |
| 1ab | 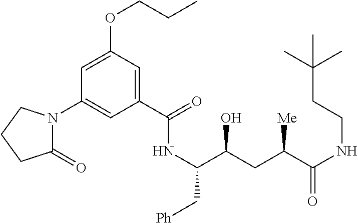 [SMILES: CCCOc1cc(C(=O)N[C@@H](Cc2ccccc2)[C@@H](O)C[C@@H](C)C(=O)NCCC(C)(C)C)cc(N2CCCC2=O)c1] |
| 1ac | 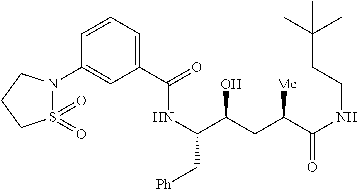 [SMILES: C[C@H](C[C@H](O)[C@H](Cc1ccccc1)NC(=O)c1cccc(N2CCCS2(=O)=O)c1)C(=O)NCCC(C)(C)C] |
| 1ad | 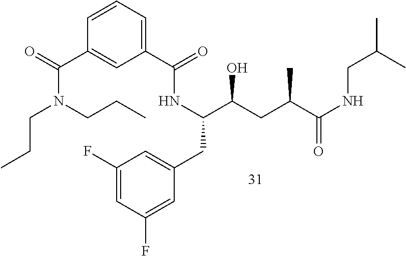 [SMILES: CCCN(CCC)C(=O)c1cccc(C(=O)N[C@@H](Cc2cc(F)cc(F)c2)[C@@H](O)C[C@@H](C)C(=O)NCC(C)C)c1] |
| 1ae | 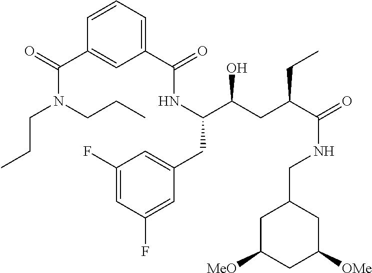 [SMILES: CCCN(CCC)C(=O)c1cccc(C(=O)N[C@@H](Cc2cc(F)cc(F)c2)[C@@H](O)C[C@@H](CC)C(=O)NCC2C[C@@H](OC)C[C@@H](OC)C2)c1] |
| 1af | 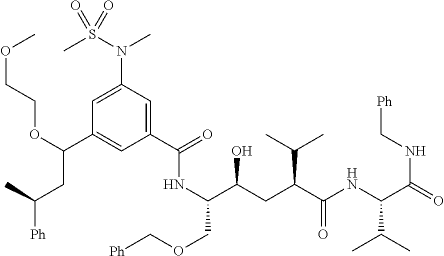 [SMILES: COCCOC(C[C@H](C)c1ccccc1)c1cc(C(=O)N[C@@H](COCc2ccccc2)[C@@H](O)C[C@H](C(=O)N[C@H](C(=O)NCc2ccccc2)C(C)C)C(C)C)cc(N(C)S(C)(=O)=O)c1] |
| 1ag | 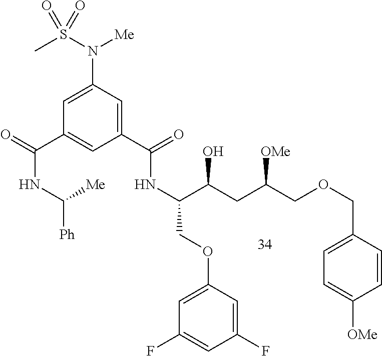 [SMILES: COc1ccc(COC[C@@H](C[C@H](O)[C@H](COc2cc(F)cc(F)c2)NC(=O)c2cc(C(=O)N[C@H](C)c3ccccc3)cc(N(C)S(C)(=O)=O)c2)OC)cc1] |
| 1ah | 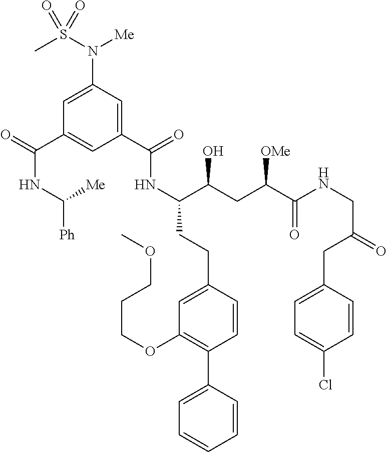 [SMILES: COCCCOc1cc(CC[C@H](NC(=O)c2cc(C(=O)N[C@H](C)c3ccccc3)cc(N(C)S(C)(=O)=O)c2)[C@@H](O)C[C@@H](OC)C(=O)NCC(=O)Cc2ccc(Cl)cc2)ccc1-c1ccccc1] |
| 1ai | 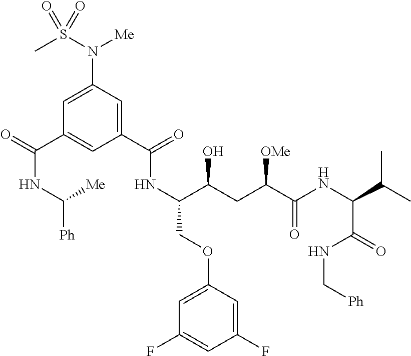 [SMILES: CO[C@H](C[C@H](O)[C@H](COc1cc(F)cc(F)c1)NC(=O)c1cc(C(=O)N[C@H](C)c2ccccc2)cc(N(C)S(C)(=O)=O)c1)C(=O)N[C@H](C(=O)NCc1ccccc1)C(C)C] |

TABLE 1d

| Compound # | Structure | Reference |
|---|---|---|
| 1 | 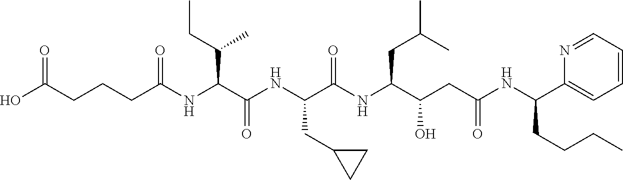 [SMILES: CCCC[C@@H](NC(=O)C[C@H](O)[C@H](CC(C)C)NC(=O)[C@H](CC1CC1)NC(=O)[C@@H](NC(=O)CCCC(=O)O)[C@@H](C)CC)c1ccccn1] | WO2006/050861 |
| 2 | 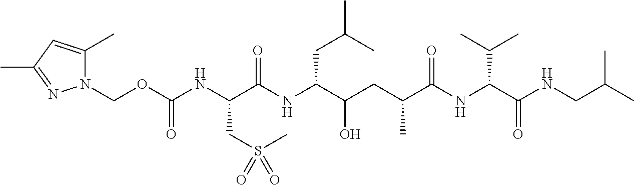 [SMILES: Cc1cc(C)n(COC(=O)N[C@@H](CS(C)(=O)=O)C(=O)N[C@H](CC(C)C)C(O)C[C@@H](C)C(=O)N[C@@H](C(=O)NCC(C)C)C(C)C)n1] | U.S. Pat. No. 7,335,632 |
| 3 | 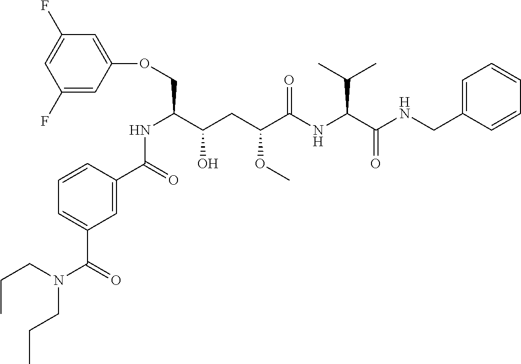 [SMILES: CCCN(CCC)C(=O)c1cccc(C(=O)N[C@@H](COc2cc(F)cc(F)c2)[C@@H](O)C[C@@H](OC)C(=O)N[C@H](C(=O)NCc2ccccc2)C(C)C)c1] | WO2008/119773 WO2008/119772 |
| 4 | 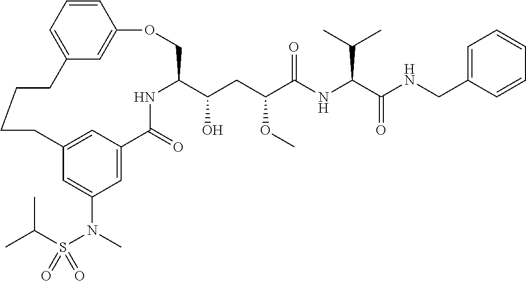 [SMILES: CO[C@H](C[C@H](O)[C@@H]1COc2cccc(c2)CCCCc2cc(cc(N(C)S(=O)(=O)C(C)C)c2)C(=O)N1)C(=O)N[C@H](C(=O)NCc1ccccc1)C(C)C] | WO2008/135488 |

TABLE 1e

| Com- | CAS | | |
|---|---|---|---|
| pound | no | Structure | Name |
| 4a | 350228- 37-4 | 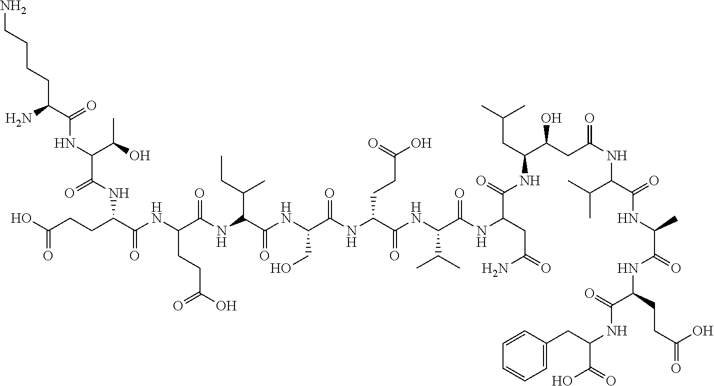 | StaVal P10- P4′ |
| 4b | 314266- 76-7 | 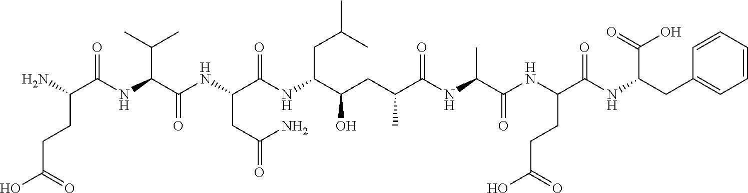 [SMILES: CC(C)C[C@@H](NC(=O)[C@H](CC(N)=O)NC(=O)[C@@H](NC(=O)[C@@H](N)CCC(=O)O)C(C)C)[C@H](O)C[C@@H](C)C(=O)N[C@@H](C)C(=O)NC(CCC(=O)O)C(=O)N[C@@H](Cc1ccccc1)C(=O)O] | OM99- 2 |
| 4c | 527674- 72-2 | 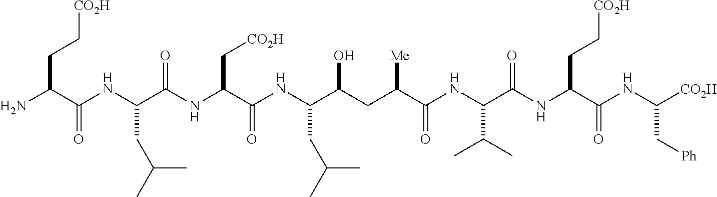 [SMILES: CC(C)C[C@H](NC(=O)[C@@H](N)CCC(=O)O)C(=O)N[C@@H](CC(=O)O)C(=O)N[C@@H](CC(C)C)[C@@H](O)C[C@@H](C)C(=O)N[C@H](C(=O)N[C@@H](CCC(=O)O)C(=O)N[C@@H](Cc1ccccc1)C(=O)O)C(C)C] | OM00- 3 |

### 3.3.1.4 Hydroxyethylamine-Based Inhibitors

In one embodiment a peptidomimetic-based inhibitor is a hydroxyethylamine-based inhibitor. Examples of these inhibitors are shown in Table 1f.

TABLE 1f

| Compound # | Structure | Reference |
|---|---|---|
| 5 | 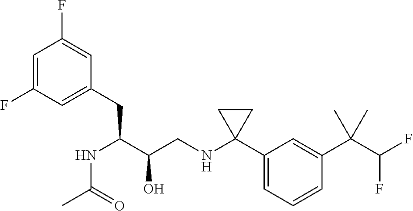 [SMILES: CC(=O)N[C@@H](Cc1cc(F)cc(F)c1)[C@H](O)CNC1(c2cccc(C(C)(C)C(F)F)c2)CC1] | WO2007/047306 WO2007/047305 WO2006/010095 WO2007/010094 U.S. Pat. No. 7,385,085 |
| 6 | 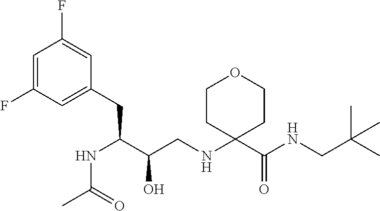 [SMILES: CC(=O)N[C@@H](Cc1cc(F)cc(F)c1)[C@H](O)CNC1(C(=O)NCC(C)(C)C)CCOCC1] | WO2007/110727 |
| 7 | 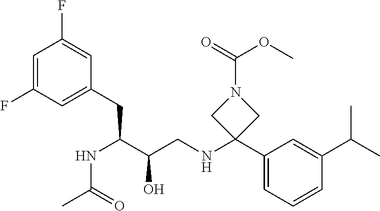 [SMILES: COC(=O)N1CC(NC[C@@H](O)[C@H](Cc2cc(F)cc(F)c2)NC(C)=O)(c2cccc(C(C)C)c2)C1] | WO2007/060526 WO2006/064324 |
| 8 | 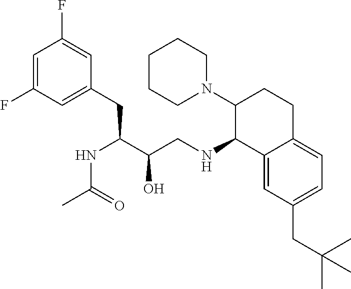 [SMILES: CC(=O)N[C@@H](Cc1cc(F)cc(F)c1)[C@H](O)CN[C@@H]1c2cc(CC(C)(C)C)ccc2CCC1N1CCCCC1] | WO2006/085216 |
| 9 | 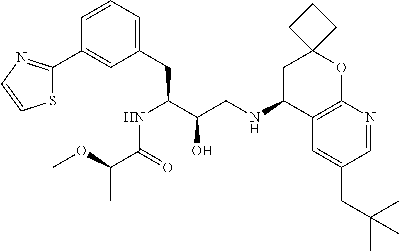 [SMILES: CO[C@H](C)C(=O)N[C@@H](Cc1cccc(-c2nccs2)c1)[C@H](O)CN[C@H]1CC2(CCC2)Oc2ncc(CC(C)(C)C)cc21] | WO2008/147547 WO2009/064418 WO2008/147544 WO2007/062007 WO2007/061930 WO2007/061670 U.S. Pat. No. 7,872,009 U.S. Pat. No. 7,803,809 U.S. Pat. No. 7,745,484 U.S. Pat. No. 7,838,676 |
| 10 | 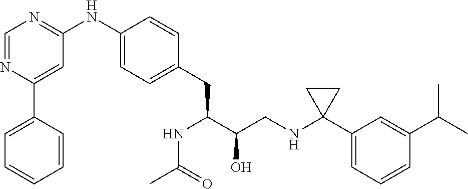 [SMILES: CC(=O)N[C@@H](Cc1ccc(Nc2cc(-c3ccccc3)ncn2)cc1)[C@H](O)CNC1(c2cccc(C(C)C)c2)CC1] | WO2008/062044 |
| 11 | 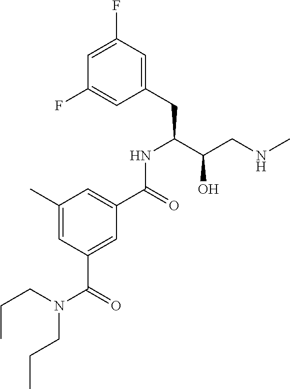 [SMILES: CCCN(CCC)C(=O)c1cc(C)cc(C(=O)N[C@@H](Cc2cc(F)cc(F)c2)[C@H](O)CNC)c1] | WO2006/032999 WO2006/033000 |
| 12 | 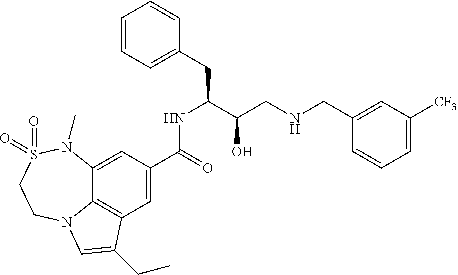 [SMILES: CCc1cn2c3c(cc(C(=O)N[C@@H](Cc4ccccc4)[C@H](O)CNCc4cccc(C(F)(F)F)c4)cc13)N(C)S(=O)(=O)CC2] | WO2006/103088 WO2006/040151 |
| 13 | 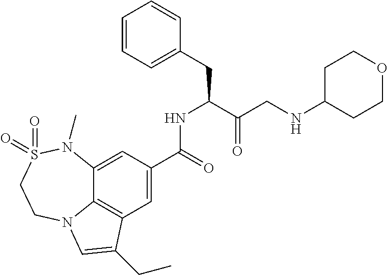 [SMILES: CCc1cn2c3c(cc(C(=O)N[C@@H](Cc4ccccc4)C(=O)CNC4CCOCC4)cc13)N(C)S(=O)(=O)CC2] | WO2006/040148 WO2006/040149 |
| 14 | 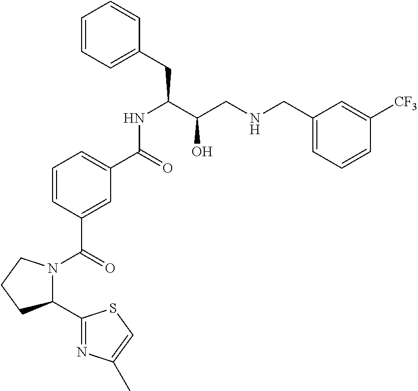 [SMILES: Cc1csc([C@H]2CCCN2C(=O)c2cccc(C(=O)N[C@@H](Cc3ccccc3)[C@H](O)CNCc3cccc(C(F)(F)F)c3)c2)n1] | WO2009/042694 WO2006/110668 WO2009/015369 |
| 15 | 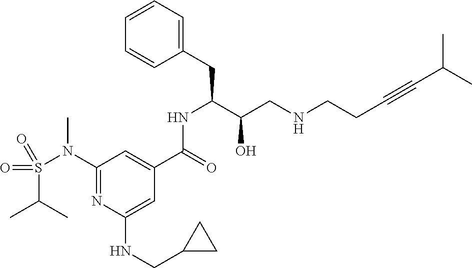 [SMILES: CC(C)C#CCCNC[C@@H](O)[C@H](Cc1ccccc1)NC(=O)c1cc(NCC2CC2)nc(N(C)S(=O)(=O)C(C)C)c1] | WO2010/107384 WO2010042030 |
| 16 | 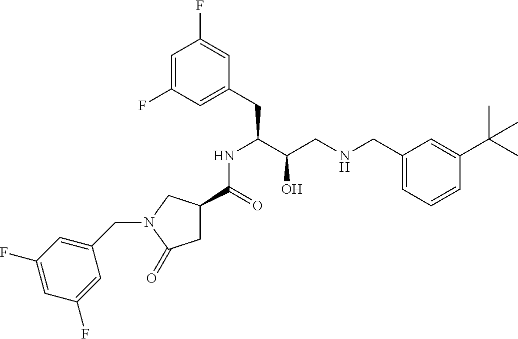 [SMILES: CC(C)(C)c1cccc(CNC[C@@H](O)[C@H](Cc2cc(F)cc(F)c2)NC(=O)[C@H]2CC(=O)N(Cc3cc(F)cc(F)c3)C2)c1] | WO2009/038411 WO2009/038412 |
| 17 | 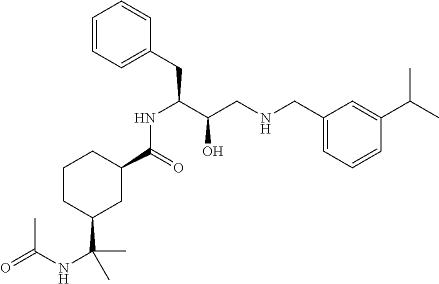 [SMILES: CC(=O)NC(C)(C)[C@H]1CCC[C@@H](C(=O)N[C@@H](Cc2ccccc2)[C@H](O)CNCc2cccc(C(C)C)c2)C1] | WO2009/013293 |

### 3.3.1.5 Cyclic and Macrocyclic Hydroxyethylenes and Hydroxyethylamines

In one embodiment, a peptidomimetic-based inhibitor is a cyclic or a macrocyclic hydroxyethylene-based. or hydroxyethylamine-based inhibitor. Examples of these inhibitors are shown in Table 1g.

TABLE 1g

| Compound # | Structure | Reference |
|---|---|---|
| 18a | 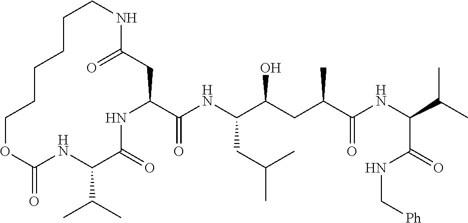 [SMILES: CC(C)C[C@H](NC(=O)[C@@H]1CC(=O)NCCCCCCOC(=O)N[C@@H](C(C)C)C(=O)N1)[C@@H](O)C[C@@H](C)C(=O)N[C@H](C(=O)NCc1ccccc1)C(C)C] | |
| 18b | 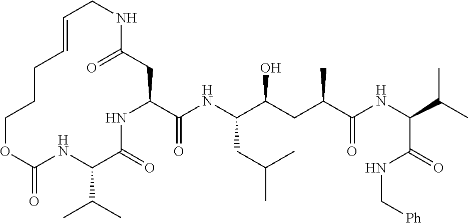 [SMILES: CC(C)C[C@H](NC(=O)[C@@H]1CC(=O)NC/C=C/CCCOC(=O)N[C@@H](C(C)C)C(=O)N1)[C@@H](O)C[C@@H](C)C(=O)N[C@H](C(=O)NCc1ccccc1)C(C)C] | |
| 18c | 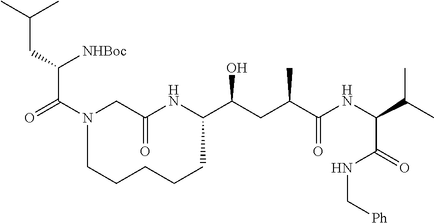 [SMILES: CC(C)C[C@H](NC(=O)OC(C)(C)C)C(=O)N1CCCCC[C@@H]([C@@H](O)C[C@@H](C)C(=O)N[C@H](C(=O)NCc2ccccc2)C(C)C)NC(=O)C1] | |
| 18d | 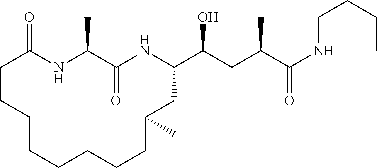 [SMILES: CCCCNC(=O)[C@H](C)C[C@H](O)[C@@H]1C[C@@H](C)CCCCCCCCC(=O)N[C@@H](C)C(=O)N1] | |
| 18 | 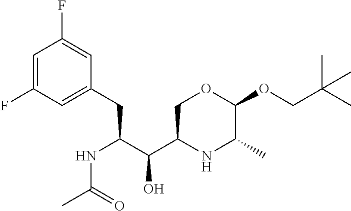 [SMILES: CC(=O)N[C@@H](Cc1cc(F)cc(F)c1)[C@H](O)[C@H]1CO[C@@H](OCC(C)(C)C)[C@H](C)N1] | WO2006/034093 |
| 19 | 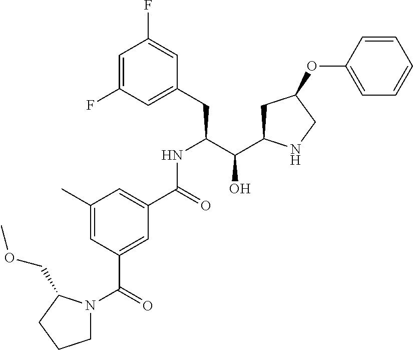 [SMILES: COC[C@H]1CCCN1C(=O)c1cc(C)cc(C(=O)N[C@@H](Cc2cc(F)cc(F)c2)[C@H](O)[C@H]2C[C@@H](Oc3ccccc3)CN2)c1] | WO2006/014762 U.S. Pat. No. 7,662,816 U.S. Pat. No. 7,598,250 |
| 20 | 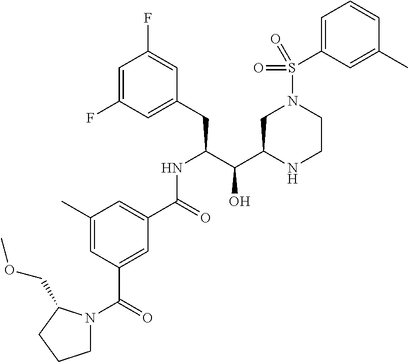 | WO2006/014762 U.S. Pat. No. 7,662,816 U.S. Pat. No. 7,598,250 |
| 21 | 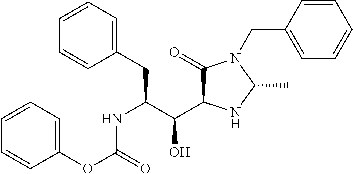 [SMILES: C[C@@H]1N[C@@H]([C@@H](O)[C@H](Cc2ccccc2)NC(=O)Oc2ccccc2)C(=O)N1Cc1ccccc1] | WO2007/058862 |
| 22 | 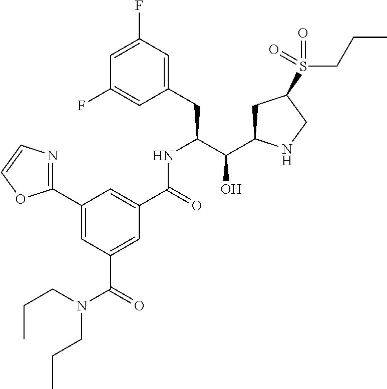 [SMILES: CCCN(CCC)C(=O)c1cc(C(=O)N[C@@H](Cc2cc(F)cc(F)c2)[C@H](O)[C@H]2C[C@@H](S(=O)(=O)CCC)CN2)cc(-c2ncco2)c1] | WO2006/099352 |
| 23 | 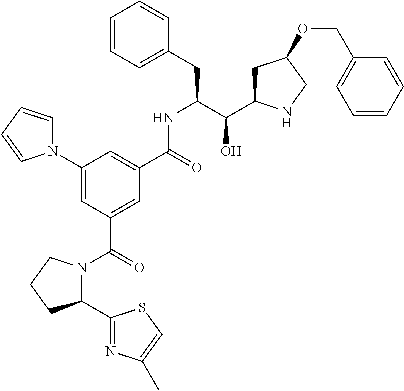 [SMILES: Cc1csc([C@H]2CCCN2C(=O)c2cc(C(=O)N[C@@H](Cc3ccccc3)[C@H](O)[C@H]3C[C@@H](OCc4ccccc4)CN3)cc(-n3cccc3)c2)n1] | WO2010/110817 WO2011/044057 WO2011/130383 WO2010/042892 WO2010/042796 |
| 24 | 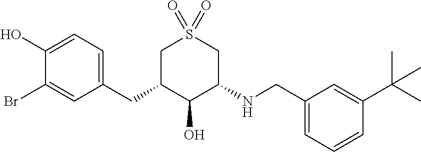 [SMILES: CC(C)(C)c1cccc(CN[C@H]2CS(=O)(=O)C[C@@H](Cc3ccc(O)c(Br)c3)[C@@H]2O)c1] | WO2010/003976 WO2009/024615 WO2007/093621 |
| 25 | 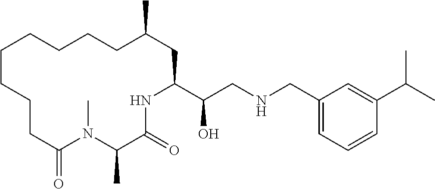 [SMILES: CC(C)c1cccc(CNC[C@@H](O)[C@@H]2C[C@H](C)CCCCCCCCC(=O)N(C)[C@H](C)C(=O)N2)c1] | WO2008/009734 |
| 26 | 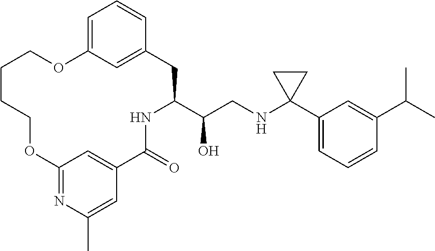 [SMILES: Cc1cc2cc(n1)OCCCCOc1cccc(c1)C[C@@H]([C@H](O)CNC1(c3cccc(C(C)C)c3)CC1)NC2=O] | WO2007/077004 WO2006/074950 |
| 27 | 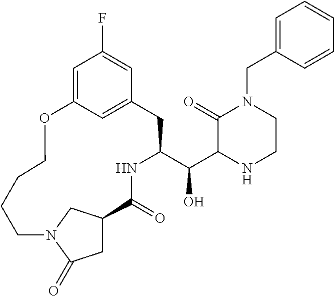 [SMILES: O=C1N[C@H]([C@H](O)C2NCCN(Cc3ccccc3)C2=O)Cc2cc(F)cc(c2)OCCCCN2C[C@@H]1CC2=O] | WO2006/014944 |

### 3.3.1.6 Carbinamines

In one embodiment, a peptidomimetic-based inhibitor is a carbinamine (primary amine)-based inhibitor. Examples of these inhibitors are shown in Table 1h.

TABLE 1h

| Compound # | Structure | Reference |
|---|---|---|
| 28 | 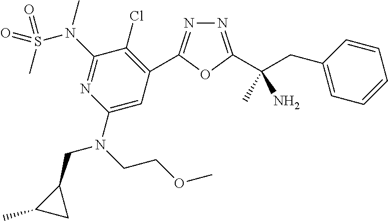 [SMILES: COCCN(C[C@H]1C[C@@H]1C)c1cc(-c2nnc([C@](C)(N)Cc3ccccc3)o2)c(Cl)c(N(C)S(C)(=O)=O)n1] | WO2006/057945 |
| 29 | 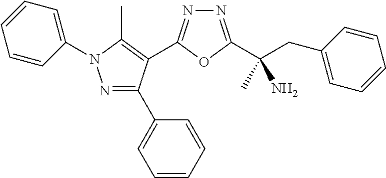 [SMILES: Cc1c(-c2nnc([C@](C)(N)Cc3ccccc3)o2)c(-c2ccccc2)nn1-c1ccccc1] | WO2006/078577 WO2006/078576 WO2007/019080 WO2007/019078 WO2007/019111 |
| 30 | 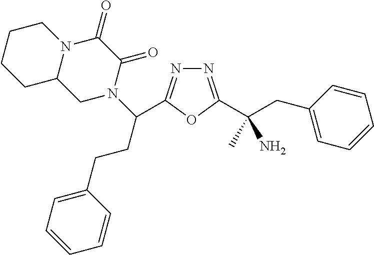 [SMILES: C[C@@](N)(Cc1ccccc1)c1nnc(C(CCc2ccccc2)N2CC3CCCCN3C(=O)C2=O)o1] | WO2006/078577 WO2006/078576 WO2007/019080 WO2007/019078 WO2007/019111 |
| 31 | 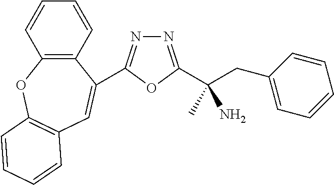 [SMILES: C[C@@](N)(Cc1ccccc1)c1nnc(C2=Cc3ccccc3Oc3ccccc32)o1] | WO2006/078577 WO2006/078576 WO2007/019080 WO2007/019078 WO2007/019111 |
| 32 | 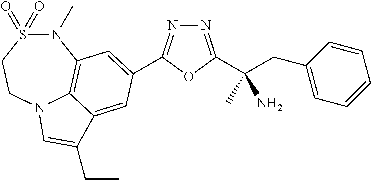 [SMILES: CCc1cn2c3c(cc(-c4nnc([C@](C)(N)Cc5ccccc5)o4)cc13)N(C)S(=O)(=O)CC2] | WO2006/078577 WO2006/078576 WO2007/019080 WO2007/019078 WO2007/019111 |
| 33 | 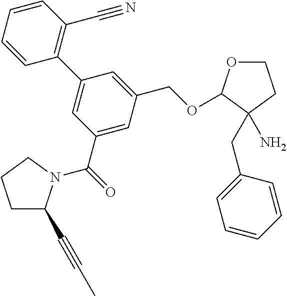 [SMILES: CC#C[C@H]1CCCN1C(=O)c1cc(COC2OCCC2(N)Cc2ccccc2)cc(-c2ccccc2C#N)c1] | WO2006/078577 WO2006/078576 WO2007/019080 WO2007/019078 WO2007/019111 |
| 34 | 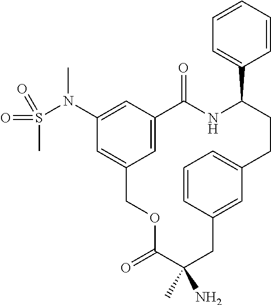 [SMILES: CN(c1cc2cc(c1)C(=O)N[C@@H](c1ccccc1)CCc1cccc(c1)C[C@@](C)(N)C(=O)OC2)S(C)(=O)=O] | WO2006/055434 |
| 35 | 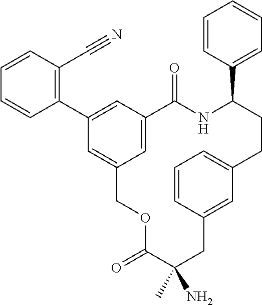 [SMILES: C[C@@]1(N)Cc2cccc(c2)CC[C@H](c2ccccc2)NC(=O)c2cc(cc(-c3ccccc3C#N)c2)COC1=O] | WO2006/055434 |
| 36 | 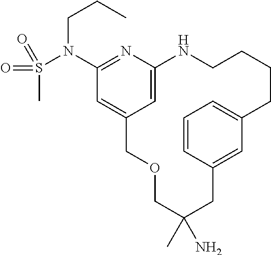 [SMILES: CCCN(c1cc2cc(n1)NCCCCc1cccc(c1)CC(C)(N)COC2)S(C)(=O)=O] | WO2006/057983 |

### 3.3.1.7. Secondary Amines

In one embodiment, a peptidomimetic-based inhibitor is a secondary amine-based inhibitor, one example being a pyrrolidine-based inhibitor. Examples of these inhibitors are shown in Table 1i.

TABLE 1i

| Compound # | Structure | Reference |
|---|---|---|
| 37 | 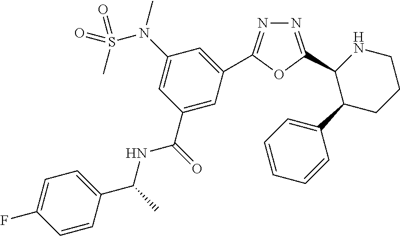 [SMILES: C[C@@H](NC(=O)c1cc(-c2nnc([C@H]3NCCC[C@H]3c3ccccc3)o2)cc(N(C)S(C)(=O)=O)c1)c1ccc(F)cc1] | WO2008/036316 |
| 38 | 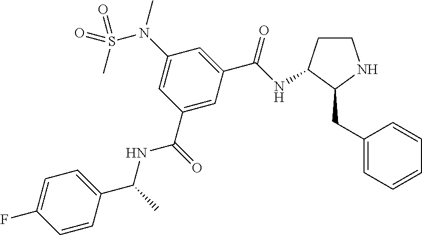 [SMILES: C[C@@H](NC(=O)c1cc(C(=O)N[C@@H]2CCN[C@H]2Cc2ccccc2)cc(N(C)S(C)(=O)=O)c1)c1ccc(F)cc1] | WO2006/002004 |
| 39 | 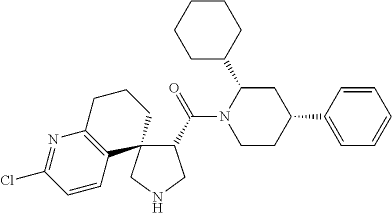 | WO2009/078932 WO2010/094242 |
| 40 | 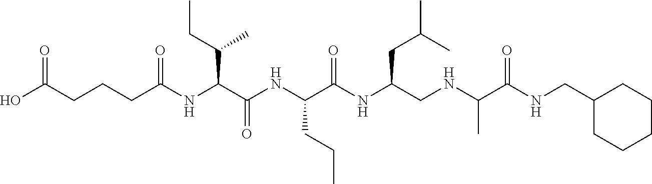 [SMILES: CCC[C@H](NC(=O)[C@@H](NC(=O)CCCC(=O)O)[C@@H](C)CC)C(=O)N[C@H](CNC(C)C(=O)NCC1CCCCC1)CC(C)C] | WO2007/014946 |
| 41 | 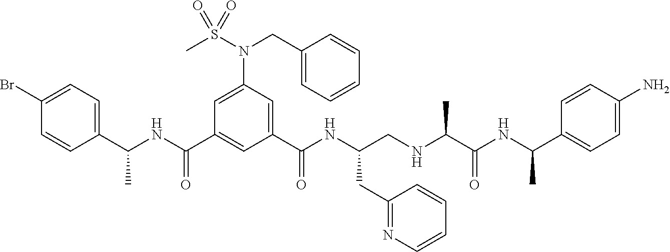 [SMILES: C[C@H](NC[C@H](Cc1ccccn1)NC(=O)c1cc(C(=O)N[C@H](C)c2ccc(Br)cc2)cc(N(Cc2ccccc2)S(C)(=O)=O)c1)C(=O)N[C@H](C)c1ccc(N)cc1] | WO2007/017510 WO2007/017509 WO2007/017507 WO2006/103038 |
| 42 | 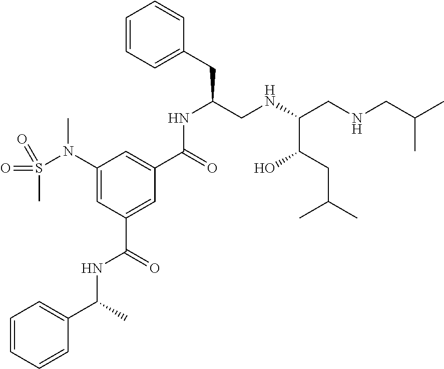 [SMILES: CC(C)CNC[C@@H](NC[C@H](Cc1ccccc1)NC(=O)c1cc(C(=O)N[C@H](C)c2ccccc2)cc(N(C)S(C)(=O)=O)c1)[C@@H](O)CC(C)C] | WO2010/065861 |

### 3.3.1.8 Tertiary Amines

In one embodiment, a peptidomimetic-based inhibitor is a tertiary amine (reduced amide)-based inhibitor. Examples of these inhibitors are shown in Table 1j.

TABLE 1j

| Com- | | |
|---|---|---|
| pound | | |
| # | Structure | Reference |
| 43 | 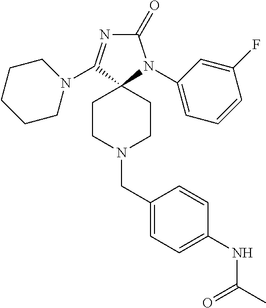 | WO2006/044497 WO2007/011810 WO2007/011833 |
| 44 | 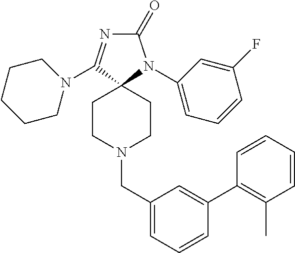 | WO2006/044497 WO2007/011810 WO2007/011833 |
| 45 | 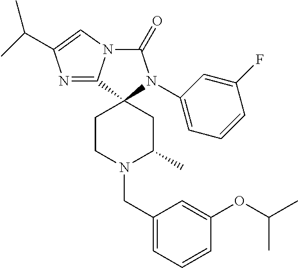 [SMILES: CC(C)Oc1cccc(CN2CC[C@@]3(C[C@@H]2C)c2nc(C(C)C)cn2C(=O)N3c2cccc(F)c2)c1] | WO2008/085509 |
| 46 | 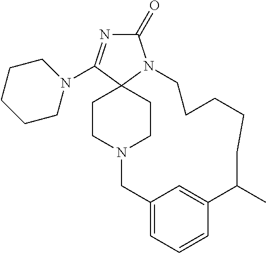 [SMILES: CC1CCCCCN2C(=O)N=C(N3CCCCC3)C23CCN(CC3)Cc2cccc1c2] | WO2008/045250 |
| 47 | 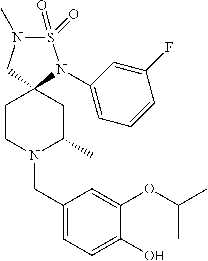 [SMILES: CC(C)Oc1cc(CN2CC[C@@]3(C[C@@H]2C)CN(C)S(=O)(=O)N3c2cccc(F)c2)ccc1O] | WO2009/136350 |
| 48 | 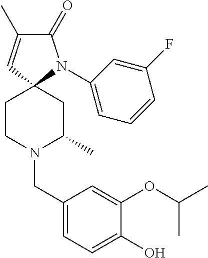 [SMILES: CC1=C[C@]2(CCN(Cc3ccc(O)c(OC(C)C)c3)[C@@H](C)C2)N(c2cccc(F)c2)C1=O] | WO2011/125006 WO2010/058333 |
| 49 | 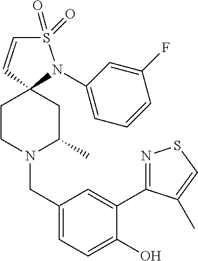 [SMILES: Cc1csnc1-c1cc(CN2CC[C@]3(C=CS(=O)(=O)N3c3cccc(F)c3)C[C@@H]2C)ccc1O] | WO2011/125006 WO2010/058333 |

### 3.3.2 Non Peptide Inhibitors

Nonpeptide inhibitors are small molecules obtainable from high throughput screening or fragment based screening of scaffolds, followed by chemical optimization. Inhibitors can also be developed based on computational screening and modeling methods, which can narrow a large compound library down to a few hundred compounds. Some isolated natural products have also been shown to have BACE1 inhibitory properties. Small molecule nonpeptide inhibitors are smaller in size, have less peptide character, and have better metabolic stability that peptidomimetic inhibitors. These small molecule inhibitors may have a lower Pgp efflux ratio.

Small molecule inhibitors for use in the invention inhibit the enzymatic action of β secretases especially in adipocytes and fat tissue. Therefore, it is not essential that a small molecule inhibitor for use in the invention should be capable of crossing the blood brain barrier (BBB). In one embodiment, a small molecule inhibitor for use as a β secretase inhibitor according to the invention does not cross the BBB or has limited capacity to cross the BBB.

Examples of non peptide, small molecule inhibitors contemplated for use as β secretase inhibitors according to the invention may be acyl-guanine-based inhibitors; 2-amino pyridine-based inhibitors; amino-imidazole-based inhibitors; amino/imino hydantoin-based inhibitors; aminothiazoline and aminooxazoline-based inhibitors; dihydroquinalzoline-based inhibitors; aminoquinoline-based inhibitors; pyrrolidine-based inhibitors; and macrocyclic non peptide-based inhibitors, as shown below:

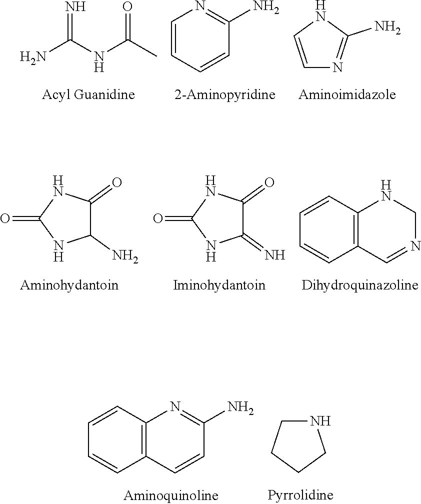

### 3.3.2.1 Arylamino and Related Compounds

In one embodiment, a small molecule inhibitor is an arylamino or related compound, for example guanidine, acyl guanidine, aminioimidazole, aminoquinoline or dihydroquinazoline. Examples of these inhibitors are shown in Table 2a.

TABLE 2a

| Compound # | Structure | Reference |
|---|---|---|
| 50 | 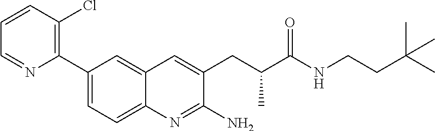 [SMILES: C[C@H](Cc1cc2cc(-c3ncccc3Cl)ccc2nc1N)C(=O)NCCC(C)(C)C] | WO2011/063233 WO2011/063272 WO2011/030311 |
| 51 | 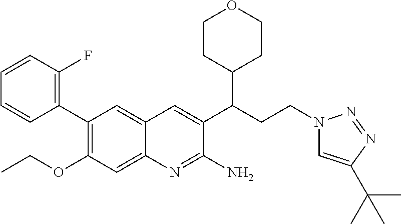 [SMILES: CCOc1cc2nc(N)c(C(CCn3cc(C(C)(C)C)nn3)C3CCOCC3)cc2cc1-c1ccccc1F] | WO2009/037401 WO2009/097278 WO2007/092854 WO2007/092846 |
| 52 | 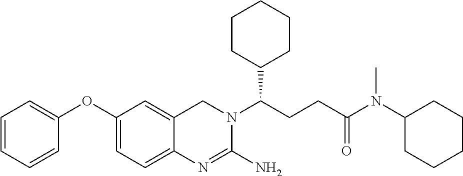 [SMILES: CN(C(=O)CC[C@@H](C1CCCCC1)N1Cc2cc(Oc3ccccc3)ccc2N=C1N)C1CCCCC1] | WO2007/050612 WO2006/024932 WO2006/017844 WO2006/017836 |
| 53 | 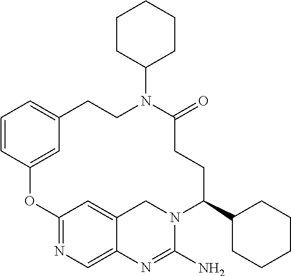 [SMILES: NC1=Nc2cnc3cc2CN1[C@H](C1CCCCC1)CCC(=O)N(C1CCCCC1)CCc1cccc(c1)O3] | WO2007/092839 |
| 54 | 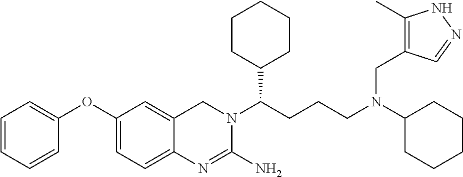 [SMILES: Cc1[nH]ncc1CN(CCC[C@@H](C1CCCCC1)N1Cc2cc(Oc3ccccc3)ccc2N=C1N)C1CCCCC1] | WO2010/059953 |
| 55 | 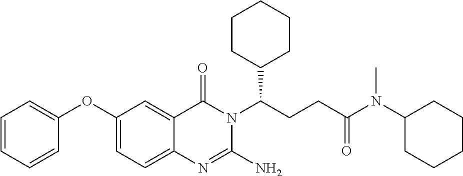 [SMILES: CN(C(=O)CC[C@@H](C1CCCCC1)n1c(N)nc2ccc(Oc3ccccc3)cc2c1=O)C1CCCCC1] | WO2009/007300 |
| 56 | 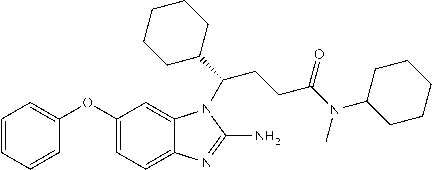 [SMILES: CN(C(=O)CC[C@@H](C1CCCCC1)n1c(N)nc2ccc(Oc3ccccc3)cc21)C1CCCCC1] | WO2009/092566 |
| 57 | 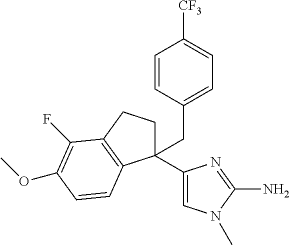 [SMILES: COc1ccc2c(c1F)CCC2(Cc1ccc(C(F)(F)F)cc1)c1cn(C)c(N)n1] | WO2009/108550 |
| 58 | 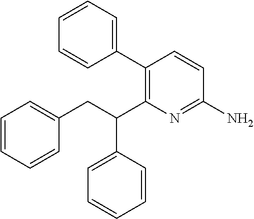 [SMILES: Nc1ccc(-c2ccccc2)c(C(Cc2ccccc2)c2ccccc2)n1] | WO2006/060109 |
| 59 | 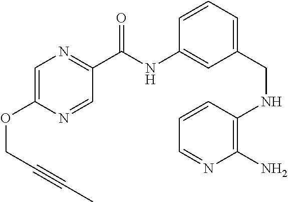 [SMILES: CC#CCOc1cnc(C(=O)Nc2cccc(CNc3cccnc3N)c2)cn1] | WO2010/047372 |
| 60 | 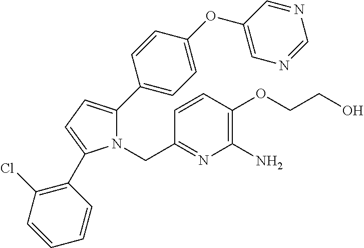 [SMILES: Nc1nc(Cn2c(-c3ccc(Oc4cncnc4)cc3)ccc2-c2ccccc2Cl)ccc1OCCO] | U.S. Pat. No. 7,732,457 WO2009/036196 |

### 3.3.2.2 Acyclic Acyl Guanidines and Related Compounds

In one embodiment, a small molecule inhibitor is an acyclic acyl guanidine or related compound, for example aryl guanidine or carbamimidate. Examples of these inhibitors are shown in Table 2b.

TABLE 2b

| Compound # | Structure | Reference |
|---|---|---|
| 61 | 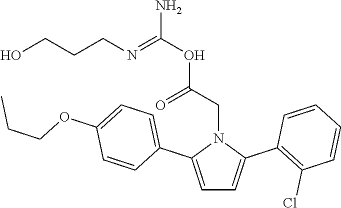 [SMILES: CCCOc1ccc(-c2ccc(-c3ccccc3Cl)n2CC(=O)O/C(N)=N/CCCO)cc1] | U.S. Pat. No. 7,488,832 U.S. Pat. No. 7,285,682 |
| 62 | 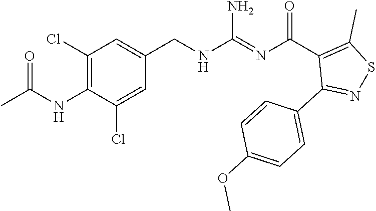 [SMILES: COc1ccc(-c2nsc(C)c2C(=O)/N=C(\N)NCc2cc(Cl)c(NC(C)=O)c(Cl)c2)cc1] | WO2007/002214 |
| 63 | 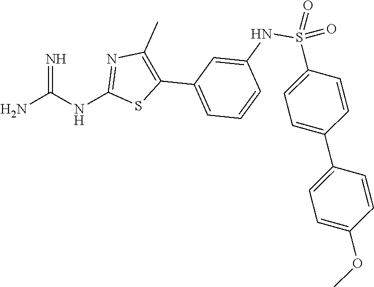 [SMILES: COc1ccc(-c2ccc(S(=O)(=O)Nc3cccc(-c4sc(NC(=N)N)nc4C)c3)cc2)cc1] | WO2007/120096 |
| 64 | 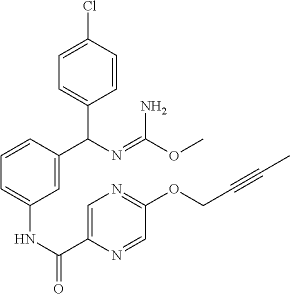 [SMILES: CC#CCOc1cnc(C(=O)Nc2cccc(C(/N=C(\N)OC)c3ccc(Cl)cc3)c2)cn1] | WO2010/113848 |

### 3.3.2.3 Amino Hydantoins and Imino Hydantoins and Related Compounds

In one embodiment, a small molecule inhibitor is an amino hydantoin or related compound, for example amino oxazoline or amino imidazoline. Examples of these inhibitors are shown in Table 2c.

TABLE 2c

| Compound # | Structure | Reference |
|---|---|---|
| 65 | 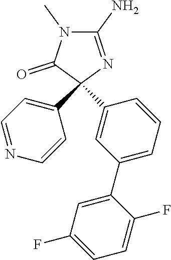 [SMILES: CN1C(=O)[C@@](c2ccncc2)(c2cccc(-c3cc(F)ccc3F)c2)N=C1N] | |
| 66 | 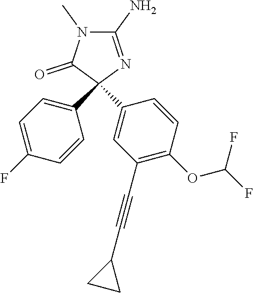 [SMILES: CN1C(=O)[C@@](c2ccc(F)cc2)(c2ccc(OC(F)F)c(C#CC3CC3)c2)N=C1N] | U.S. Pat. No. 7,452,885 |
| 67 | 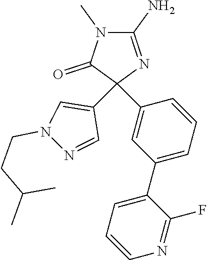 [SMILES: CC(C)CCn1cc(C2(c3cccc(-c4cccnc4F)c3)N=C(N)N(C)C2=O)cn1] | WO2008/118379 WO2008/115552 U.S. Pat. No. 7,723,368 U.S. Pat. No. 7,700,602 U.S. Pat. No. 7,482,349 U.S. Pat. No. 7,423,158 |
| 68 | 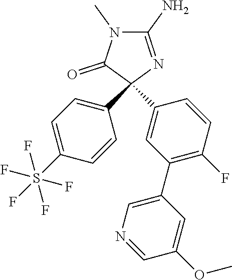 [SMILES: COc1cncc(-c2cc([C@@]3(c4ccc(S(F)(F)(F)(F)F)cc4)N=C(N)N(C)C3=O)ccc2F)c1] | U.S. Pat. No. 7,705,030 U.S. Pat. No. 7,417,047 |
| 69 | 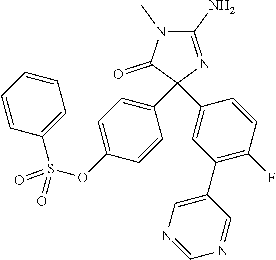 [SMILES: CN1C(=O)C(c2ccc(OS(=O)(=O)c3ccccc3)cc2)(c2ccc(F)c(-c3cncnc3)c2)N=C1N] | WO2008/076045 WO2008/076044 WO2008/076043 WO2007/058601 |
| 70 | 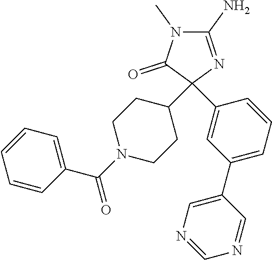 [SMILES: CN1C(=O)C(c2cccc(-c3cncnc3)c2)(C2CCN(C(=O)c3ccccc3)CC2)N=C1N] | WO2008/076046 WO2007/058602 |
| 71 | 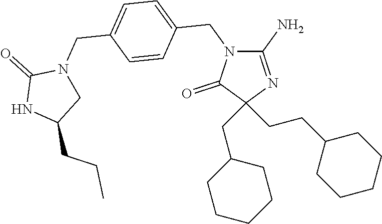 [SMILES: CCC[C@@H]1CN(Cc2ccc(CN3C(=O)C(CCC4CCCCC4)(CC4CCCCC4)N=C3N)cc2)C(=O)N1] | U.S. Pat. No. 7,700,603 U.S. Pat. No. 7,592,348 |
| 72 | 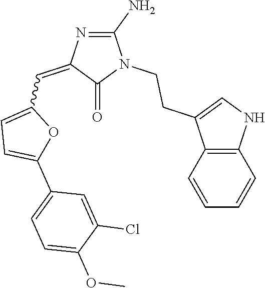 [SMILES: COc1ccc(-c2ccc(C=C3N=C(N)N(CCc4c[nH]c5ccccc45)C3=O)o2)cc1Cl] | WO2007/078813 WO2011/015646 |
| 73 | 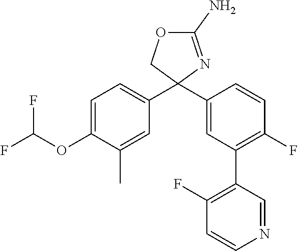 [SMILES: Cc1cc(C2(c3ccc(F)c(-c4cnccc4F)c3)COC(N)=N2)ccc1OC(F)F] | WO2009/103626 |
| 74 | 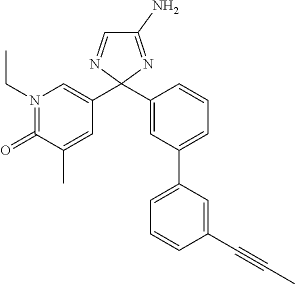 | WO2011/002408 WO2011/002407 |
| 74a | 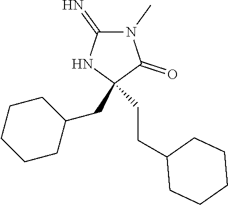 [SMILES: CN1C(=N)N[C@](CCC2CCCCC2)(CC2CCCCC2)C1=O] | |
| 74b | 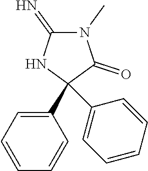 | |
| 74c | 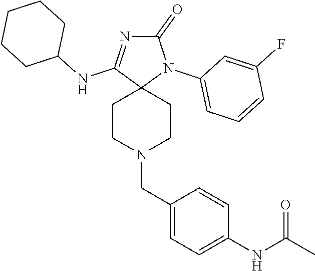 [SMILES: CC(=O)Nc1ccc(CN2CCC3(CC2)C(NC2CCCCC2)=NC(=O)N3c2cccc(F)c2)cc1] | |

### 3.3.2.4 Amino Imidazole and Related Compounds

In one embodiment, a small molecule inhibitor is an amino imidazole or related compound. Examples of these inhibitors are shown in Table 2d.

TABLE 2d

| Compound # | Structure | Reference |
|---|---|---|
| 75 | | U.S. Pat. No. 7,456,186 |
| 76 | 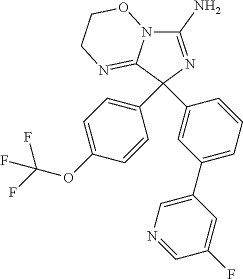 [SMILES: NC1=NC(c2ccc(OC(F)(F)F)cc2)(c2cccc(-c3cncc(F)c3)c2)C2=NCCON12] | WO2008/022024 |
| 77 | 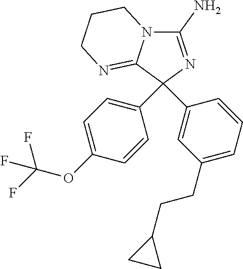 [SMILES: NC1=NC(c2ccc(OC(F)(F)F)cc2)(c2cccc(CCC3CC3)c2)C2=NCCCN12] | WO2006/076284 |
| 78 | 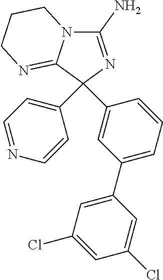 [SMILES: NC1=NC(c2ccncc2)(c2cccc(-c3cc(Cl)cc(Cl)c3)c2)C2=NCCCN12] | WO2007/145570 WO2007/145568 |
| 79 | 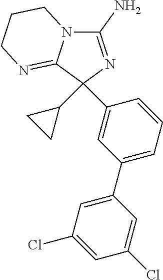 [SMILES: NC1=NC(c2cccc(-c3cc(Cl)cc(Cl)c3)c2)(C2CC2)C2=NCCCN12] | WO2008/063114 |
| 80 | 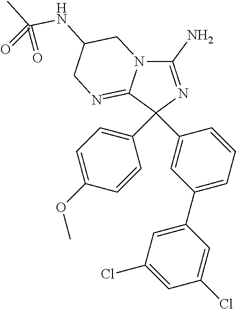 | WO2007/145571 |
| 81 | 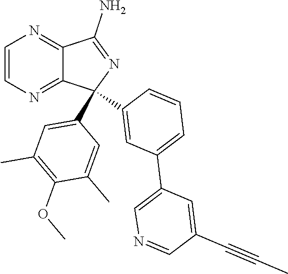 [SMILES: CC#Cc1cncc(-c2cccc([C@@]3(c4cc(C)c(OC)c(C)c4)N=C(N)c4nccnc43)c2)c1] | WO2011/002409 WO2010/056196 WO2010/056195 WO2010/056194 WO2009/005471 WO2009/005470 WO2007/149033 U.S. Pat. No. 7,629,356 |

### 3.3.2.5 Spirocyclic Hydantoins and Related Compounds

In one embodiment, a small molecule inhibitor is spirocyclic hydantoin or related compound. Examples of these inhibitors are shown in Table 2e.

TABLE 2e

| Compound # | Structure | Reference |
|---|---|---|
| 82 | 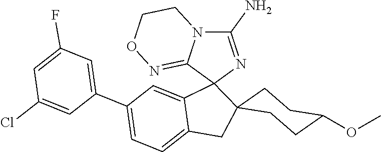 [SMILES: COC1CCC2(CC1)Cc1ccc(-c3cc(F)cc(Cl)c3)cc1C21N=C(N)N2CCON=C21] | WO2011/106414 W02010/105179 |
| 83 | 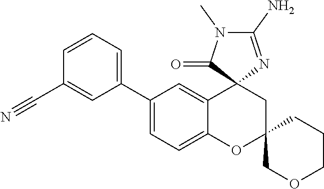 [SMILES: CN1C(=O)[C@]2(C[C@]3(CCCOC3)Oc3ccc(-c4cccc(C#N)c4)cc32)N=C1N] | WO2010/021680 |
| 84 | 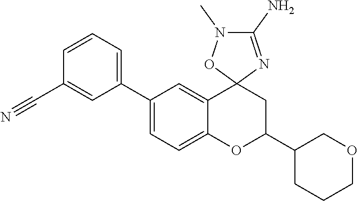 [SMILES: CN1OC2(CC(C3CCCOC3)Oc3ccc(-c4cccc(C#N)c4)cc32)N=C1N] | WO2010/021680 |
| 85 | 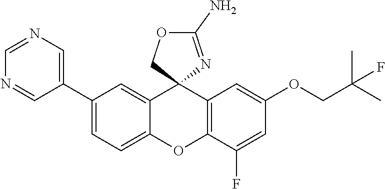 [SMILES: CC(C)(F)COc1cc(F)c2c(c1)[C@]1(COC(N)=N1)c1cc(-c3cncnc3)ccc1O2] | WO2010/030954 WO2011/115938 |
| 86 | 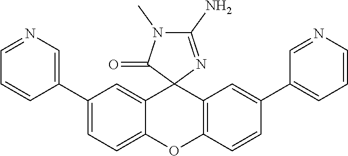 [SMILES: CN1C(=O)C2(N=C1N)c1cc(-c3cccnc3)ccc1Oc1ccc(-c3cccnc3)cc12] | WO2011/130741 |
| 87 | 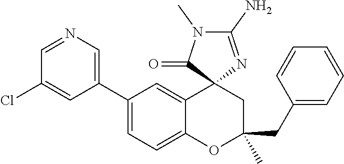 [SMILES: CN1C(=O)[C@]2(C[C@@](C)(Cc3ccccc3)Oc3ccc(-c4cncc(Cl)c4)cc32)N=C1N] | WO2011/123674 WO2011/072064 |
| 88 | 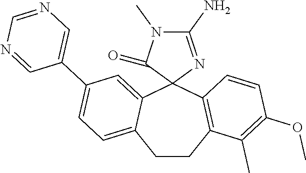 [SMILES: COc1ccc2c(c1C)CCc1ccc(-c3cncnc3)cc1C21N=C(N)N(C)C1=O] | U.S. Pat. No. 7,582,667 |

### 3.3.2.6 Cyclic Acylguanidines and Related Compounds

In one embodiment, a small molecule inhibitor is cyclic acylguanidines or related compound such as sulfonyl guanidine, iminopyrimidinone. Examples of these inhibitors are shown in Table 2f.

TABLE 2f

| Compound # | Structure | Reference |
|---|---|---|
| 89 | 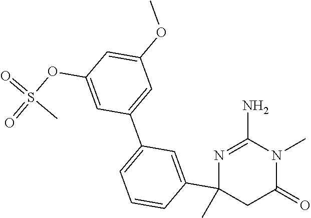 [SMILES: COc1cc(OS(C)(=O)=O)cc(-c2cccc(C3(C)CC(=O)N(C)C(N)=N3)c2)c1] | WO2007/073284 WO2006/041405 WO2006/041404 WO2007/114771 |
| 90 | 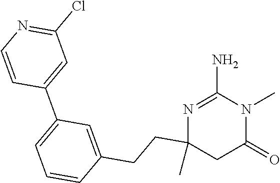 [SMILES: CN1C(=O)CC(C)(CCc2cccc(-c3ccnc(Cl)c3)c2)N=C1N] | WO2007/073284 WO2006/041405 WO2006/041404 WO2007/114771 |
| 91 | 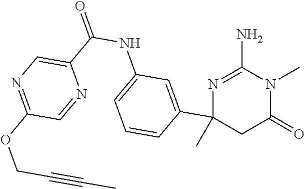 [SMILES: CC#CCOc1cnc(C(=O)Nc2cccc(C3(C)CC(=O)N(C)C(N)=N3)c2)cn1] | WO2010/047372 |
| 92 | 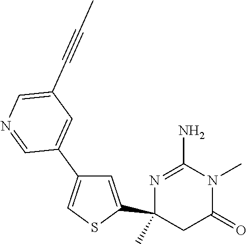 [SMILES: CC#Cc1cncc(-c2csc([C@]3(C)CC(=O)N(C)C(N)=N3)c2)c1] | U.S. Pat. No. 7,763,609 WO2008/103351 |
| 93 | 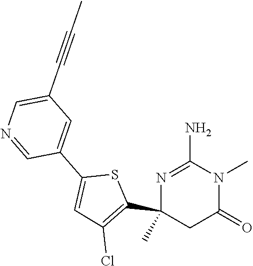 [SMILES: CC#Cc1cncc(-c2cc(Cl)c([C@]3(C)CC(=O)N(C)C(N)=N3)s2)c1] | U.S. Pat. No. 7,763,609 WO2008/103351 |
| 94 | 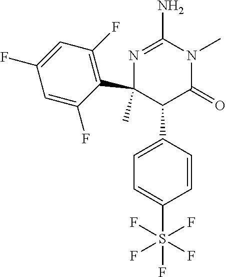 [SMILES: CN1C(=O)[C@H](c2ccc(S(F)(F)(F)(F)F)cc2)[C@@](C)(c2c(F)cc(F)cc2F)N=C1N] | W2011/044185 |
| 95 | 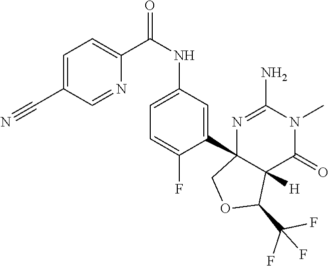 [SMILES: CN1C(=O)[C@@H]2[C@@H](C(F)(F)F)OC[C@]2(c2cc(NC(=O)c3ccc(C#N)cn3)ccc2F)N=C1N] | WO2011/009897 WO2009/131974 WO2009/131975 |
| 96 | 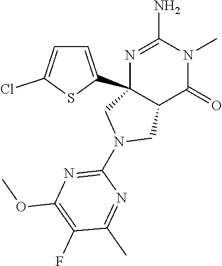 [SMILES: COc1nc(N2C[C@H]3C(=O)N(C)C(N)=N[C@@]3(c3ccc(Cl)s3)C2)nc(C)c1F] | WO2011/009897 WO2009/131974 WO2009/131975 |
| 97 | 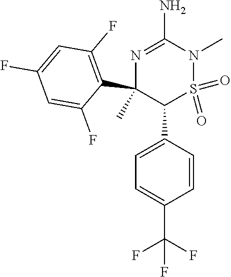 [SMILES: CN1C(N)=N[C@](C)(c2c(F)cc(F)cc2F)[C@@H](c2ccc(C(F)(F)F)cc2)S1(=O)=O] | WO2011/044187 |
| 97a | 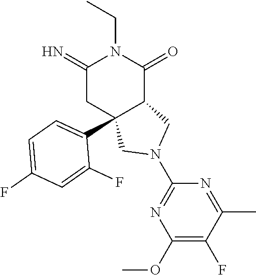 [SMILES: CCN1C(=N)C[C@@]2(c3ccc(F)cc3F)CN(c3nc(C)c(F)c(OC)n3)C[C@H]2C1=O] | |

### 3.3.2.7 Cyclic Isothioureas and Isoureas

In one embodiment, a small molecule inhibitor is cyclic isothioureas and isourea or related compound including aminothiazolines and aminooxazolines. Examples of these inhibitors are shown in Table 2g.

TABLE 2g

| Compound # | Structure | Reference |
|---|---|---|
| 98 | 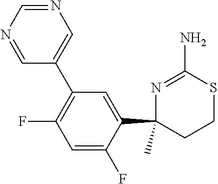 [SMILES: C[C@@]1(c2cc(-c3cncnc3)c(F)cc2F)CCSC(N)=N1] | WO2009/134617 |
| 99 | 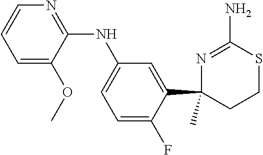 [SMILES: COc1cccnc1Nc1ccc(F)c([C@]2(C)CCSC(N)=N2)c1] | WO2011/058763 WO2007/049532 |
| 100 | 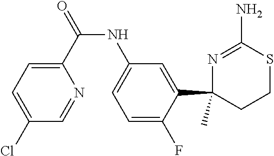 [SMILES: C[C@@]1(c2cc(NC(=O)c3ccc(Cl)cn3)ccc2F)CCSC(N)=N1] | WO2011/058763 WO2007/049532 |
| 101 | 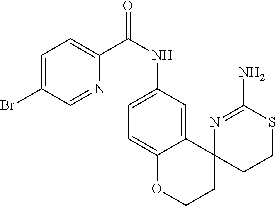 [SMILES: NC1=NC2(CCOc3ccc(NC(=O)c4ccc(Br)cn4)cc32)CCS1] | WO2010/013302 WO2010/013794 |
| 102 | 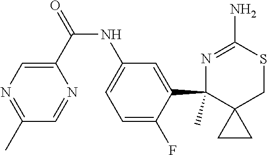 [SMILES: Cc1cnc(C(=O)Nc2ccc(F)c([C@@]3(C)N=C(N)SCC34CC4)c2)cn1] | WO2008/133274 WO2008/133273 |
| 103 | 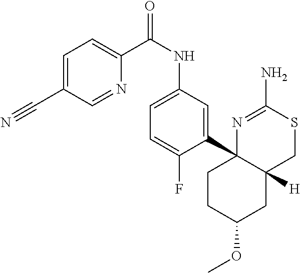 [SMILES: CO[C@@H]1CC[C@]2(c3cc(NC(=O)c4ccc(C#N)cn4)ccc3F)N=C(N)SC[C@@H]2C1] | WO2009/091016 WO2010/038686 |
| 104 | 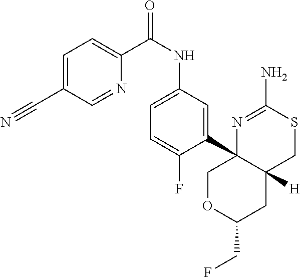 [SMILES: N#Cc1ccc(C(=O)Nc2ccc(F)c([C@]34CO[C@@H](CF)C[C@H]3CSC(N)=N4)c2)nc1] | WO2009/091016 WO2010/038686 |
| 105 | 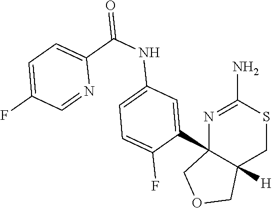 [SMILES: NC1=N[C@@]2(c3cc(NC(=O)c4ccc(F)cn4)ccc3F)COC[C@H]2CS1] | WO2011/005738 |
| 106 | 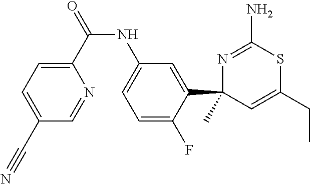 [SMILES: CCC1=C[C@@](C)(c2cc(NC(=O)c3ccc(C#N)cn3)ccc2F)N=C(N)S1] | W02011/070781 WO2011/071057 |
| 107 | 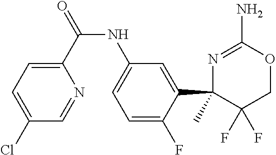 [SMILES: C[C@]1(c2cc(NC(=O)c3ccc(Cl)cn3)ccc2F)N=C(N)OCC1(F)F] | WO2011/069934 |
| 108 | 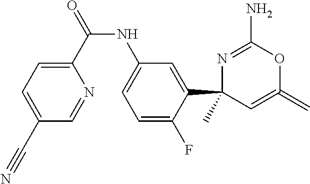 | WO2011/071135 |
| 109 | 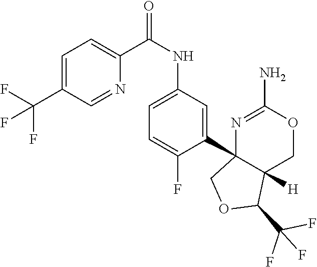 [SMILES: NC1=N[C@@]2(c3cc(NC(=O)c4ccc(C(F)(F)F)cn4)ccc3F)CO[C@H](C(F)(F)F)[C@H]2CO1] | WO2011/009898 |
| 110 | 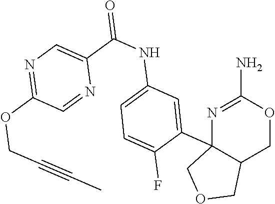 [SMILES: CC#CCOc1cnc(C(=O)Nc2ccc(F)c(C34COCC3COC(N)=N4)c2)cn1] | WO2011/071109 |
| 110a | 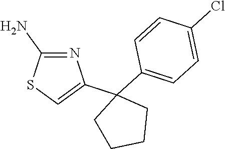 [SMILES: Nc1nc(C2(c3ccc(Cl)cc3)CCCC2)cs1] | |
| 110b | 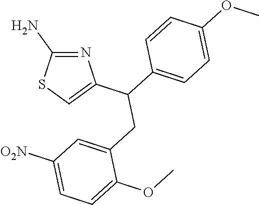 [SMILES: COc1ccc(C(Cc2cc([N+](=O)[O-])ccc2OC)c2csc(N)n2)cc1] | |
| 110c | 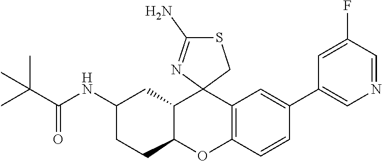 | |
| 110d | 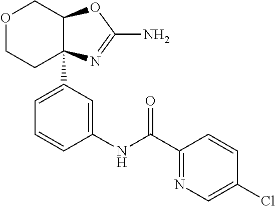 [SMILES: NC1=N[C@@]2(c3cccc(NC(=O)c4ccc(Cl)cn4)c3)CCOC[C@H]2O1] | |
| 110e | 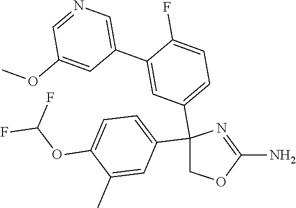 [SMILES: COc1cncc(-c2cc(C3(c4ccc(OC(F)F)c(C)c4)COC(N)=N3)ccc2F)c1] | |
| 110f | 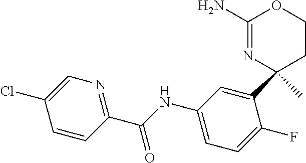 [SMILES: C[C@@]1(c2cc(NC(=O)c3ccc(Cl)cn3)ccc2F)CCOC(N)=N1] | |
| 110g | 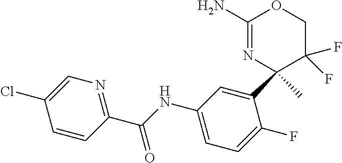 [SMILES: C[C@]1(c2cc(NC(=O)c3ccc(Cl)cn3)ccc2F)N=C(N)OCC1(F)F] | |
| 110h | 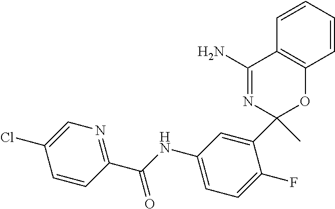 [SMILES: CC1(c2cc(NC(=O)c3ccc(Cl)cn3)ccc2F)N=C(N)c2ccccc2O1] | |
| 110i | 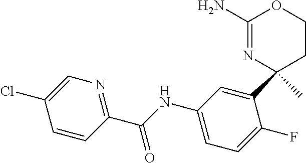 [SMILES: C[C@@]1(c2cc(NC(=O)c3ccc(Cl)cn3)ccc2F)CCOC(N)=N1] | |
| 110j | 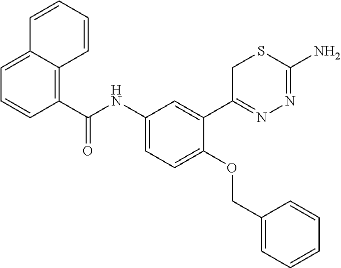 [SMILES: NC1=NN=C(c2cc(NC(=O)c3cccc4ccccc34)ccc2OCc2ccccc2)CS1] | |
| 110k | 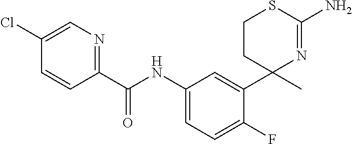 [SMILES: CC1(c2cc(NC(=O)c3ccc(Cl)cn3)ccc2F)CCSC(N)=N1] | |
| 110l | 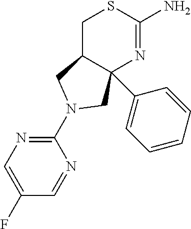 [SMILES: NC1=N[C@@]2(c3ccccc3)CN(c3ncc(F)cn3)C[C@H]2CS1] | |
| 110m | 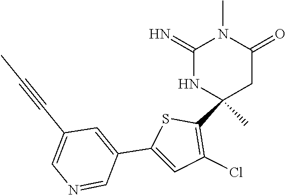 [SMILES: CC#Cc1cncc(-c2cc(Cl)c([C@]3(C)CC(=O)N(C)C(=N)N3)s2)c1] | |

### 3.3.2.8 Cyclic Amidines

In one embodiment, a small molecule inhibitor is a cyclic amidine or related compound, including for example amino oxazepines and amino thiazepines. Examples of these inhibitors are shown in Table 2h.

TABLE 2

| Compound # | Structure | Reference |
|---|---|---|
| 111 | 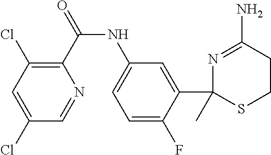 [SMILES: CC1(c2cc(NC(=O)c3ncc(Cl)cc3Cl)ccc2F)N=C(N)CCS1] | WO2011/077726 |
| 112 | 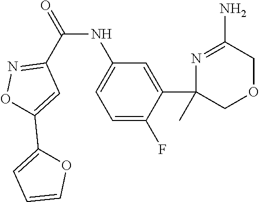 [SMILES: CC1(c2cc(NC(=O)c3cc(-c4ccco4)on3)ccc2F)COCC(N)=N1] | WO2011/020806 |
| 113 | 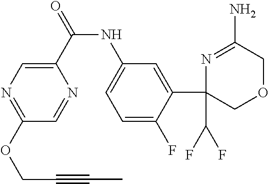 [SMILES: CC#CCOc1cnc(C(=O)Nc2ccc(F)c(C3(C(F)F)COCC(N)=N3)c2)cn1] | WO2011/080176 |
| 114 | 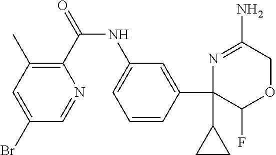 [SMILES: Cc1cc(Br)cnc1C(=O)Nc1cccc(C2(C3CC3)N=C(N)COC2F)c1] | WO2011/154431 |
| 115 | 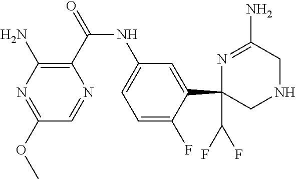 [SMILES: COc1cnc(C(=O)Nc2ccc(F)c([C@]3(C(F)F)CNCC(N)=N3)c2)c(N)n1] | WO2011/009943 |
| 116 | 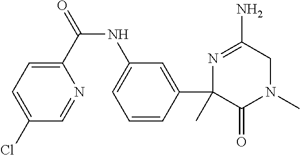 [SMILES: CN1CC(N)=NC(C)(c2cccc(NC(=O)c3ccc(Cl)cn3)c2)C1=O] | WO2011/154374 |
| 117 | 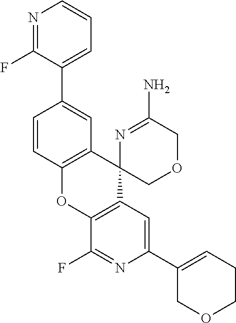 [SMILES: NC1=N[C@@]2(COC1)c1cc(-c3cccnc3F)ccc1Oc1c2cc(C2=CCCOC2)nc1F] | WO2011/115928 |
| 118 | 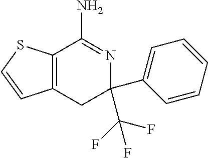 [SMILES: NC1=NC(c2ccccc2)(C(F)(F)F)Cc2ccsc21] | WO2007/058583 |
| 119 | 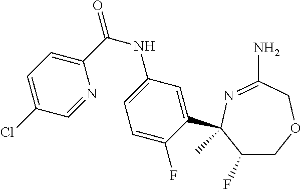 [SMILES: C[C@]1(c2cc(NC(=O)c3ccc(Cl)cn3)ccc2F)N=C(N)COC[C@@H]1F] | WO2011/138293 |
| 119a | 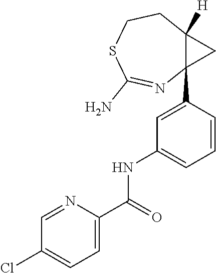 [SMILES: NC1=N[C@@]2(c3cccc(NC(=O)c4ccc(Cl)cn4)c3)C[C@H]2CCS1] | |

### 3.3.2.9 Acylbenzimidazoles and Diarylureas

In one embodiment, a small molecule inhibitor is an acylbenzimidazole or diarylurea. Examples of these inhibitors are shown in Table 2i.

TABLE 21

| Compound # | Structure | Reference |
|---|---|---|
| 120 | 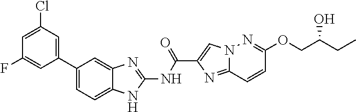 [SMILES: CC[C@@H](O)COc1ccc2nc(C(=O)Nc3nc4cc(-c5cc(F)cc(Cl)c5)ccc4[nH]3)cn2n1] | WO2011/119465 |
| 121 | 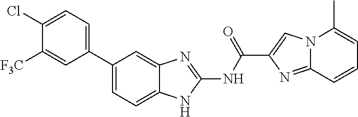 [SMILES: Cc1cccc2nc(C(=O)Nc3nc4cc(-c5ccc(Cl)c(C(F)(F)F)c5)ccc4[nH]3)cn12] | WO2010/126745 WO2010/126743 |
| 122 | 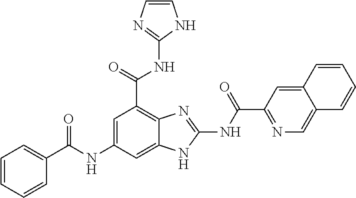 [SMILES: O=C(Nc1cc(C(=O)Nc2ncc[nH]2)c2nc(NC(=O)c3cc4ccccc4cn3)[nH]c2c1)c1ccccc1] | WO2006/099379 |
| 123 | 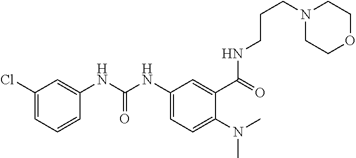 [SMILES: CN(C)c1ccc(NC(=O)Nc2cccc(Cl)c2)cc1C(=O)NCCCN1CCOCC1] | WO2006/133588 |
| 124 | 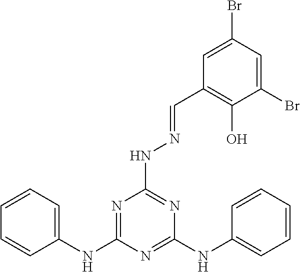 [SMILES: Oc1c(Br)cc(Br)cc1/C=N/Nc1nc(Nc2ccccc2)nc(Nc2ccccc2)n1] | WO2007/051333 |

### 3.3.3 Miscellaneous Inhibitors

Examples of compounds contemplated for use as β secretase inhibitors according to the invention are described in Table 3.

TABLE 3

| Compound | CAS no | Structure | Name |
|---|---|---|---|
| 125 | 1200493-78-2 | 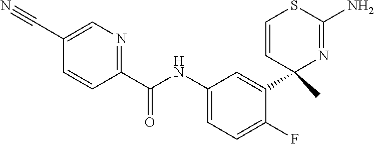 [SMILES: C[C@@]1(c2cc(NC(=O)c3ccc(C#N)cn3)ccc2F)C=CSC(N)=N1] | atabecestat |
| 126 | | 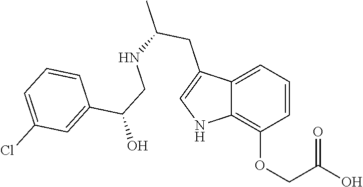 [SMILES: C[C@H](Cc1c[nH]c2c(OCC(=O)O)cccc12)NC[C@H](O)c1cccc(Cl)c1] | Vtp 37948 bi 1181181 |
| 127 | 1388651-30-6 | 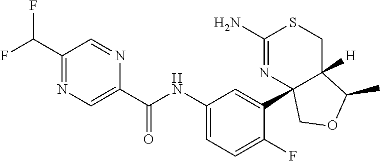 [SMILES: C[C@H]1OC[C@]2(c3cc(NC(=O)c4cnc(C(F)F)cn4)ccc3F)N=C(N)SC[C@H]12] | elenbecestat |
| 128 | 1383982-64-6 | 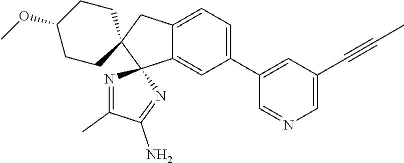 [SMILES: CC#Cc1cncc(-c2ccc3c(c2)[C@@]2(N=C(C)C(N)=N2)[C@]2(CC[C@@H](OC)CC2)C3)c1] | lanabecestat |
| 129 | 1387560-01-1 | 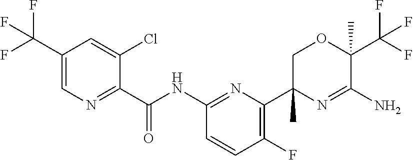 [SMILES: C[C@@]1(c2nc(NC(=O)c3ncc(C(F)(F)F)cc3Cl)ccc2F)CO[C@@](C)(C(F)(F)F)C(N)=N1] | umibecestat |
| 130 | 1286770-55-5 | 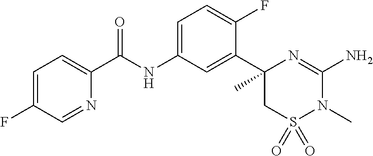 [SMILES: CN1C(N)=N[C@](C)(c2cc(NC(=O)c3ccc(F)cn3)ccc2F)CS1(=O)=O] | verubecestat |
| 131 | 1262036-50-9 | 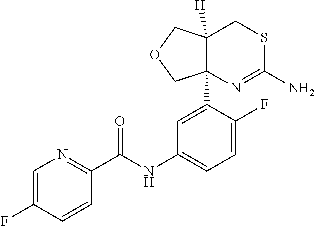 [SMILES: NC1=N[C@@]2(c3cc(NC(=O)c4ccc(F)cn4)ccc3F)COC[C@H]2CS1] | Ly2886721 |
| 132 | 1628690-73-2 | 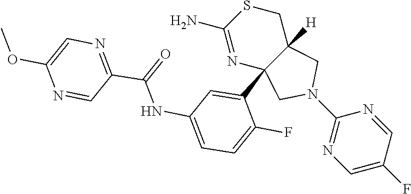 [SMILES: COc1cnc(C(=O)Nc2ccc(F)c([C@]34CN(c5ncc(F)cn5)C[C@H]3CSC(N)=N4)c2)cn1] | Ly3202626 |
| 134 | 1310347-50-2 | 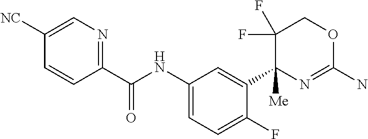 [SMILES: C[C@]1(c2cc(NC(=O)c3ccc(C#N)cn3)ccc2F)N=C(N)OCC1(F)F] | RG7129 |
| 135 | 1818339-66-0 | 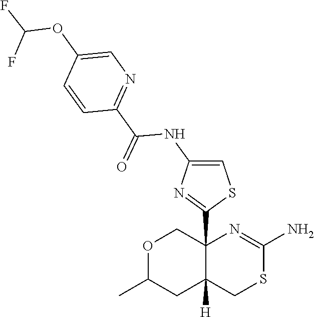 [SMILES: CC1C[C@H]2CSC(N)=N[C@@]2(c2nc(NC(=O)c3ccc(OC(F)F)cn3)cs2)CO1] | Pf-06751979 |
| 136 | 1227163-84-9 | 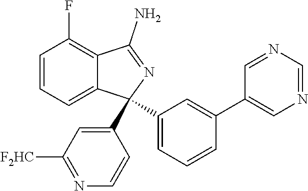 [SMILES: NC1=N[C@@](c2cccc(-c3cncnc3)c2)(c2ccnc(C(F)F)c2)c2cccc(F)c21] | AZD3839 |

### 3.4 Pharmaceutical Compositions and Administration

The β secretase inhibitors described herein and the pharmaceutically acceptable salts can be used as therapeutically active substances, e.g. in the form of pharmaceutical preparations. The pharmaceutical preparations can be administered orally, e.g. in the form of tablets, coated tablets, dragées, hard and soft gelatin capsules, solutions, emulsions or suspensions. The administration can, however, also be effected rectally, e.g. in the form of suppositories, or parenterally, e.g. in the form of injection solutions.

The β secretase inhibitors described herein and the pharmaceutically acceptable salts thereof can be processed with pharmaceutically inert, inorganic or organic carriers for the production of pharmaceutical preparations. Lactose, corn starch or derivatives thereof, talc, stearic acids or its salts and the like can be used, for example, as such carriers for tablets, coated tablets, dragees and hard gelatin capsules. Suitable carriers for soft gelatin capsules are, for example, vegetable oils, waxes, fats, semi-solid and liquid polyols and the like. Depending on the nature of the active substance no carriers are however usually required in the case of soft gelatin capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical preparations can, moreover, contain pharmaceutically acceptable auxiliary substances such as preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

Medicaments containing a β secretase inhibitor described herein and the pharmaceutically acceptable salts and a therapeutically inert carrier are also provided by the present invention, as is a process for their production, which comprises β secretase inhibitor described herein and the pharmaceutically acceptable salts and, if desired, one or more other therapeutically valuable substances into a galenical administration form together with one or more therapeutically inert carriers.

The dosage can vary within wide limits and will, of course, have to be adjusted to the individual requirements in each particular case. In the case of oral administration the dosage for adults can vary from about 0.01 mg to about 1000 mg per day of a secretase inhibitor described herein and the pharmaceutically acceptable salts. The daily dosage may be administered as single dose or in divided doses and, in addition, the upper limit can also be exceeded when this is found to be indicated.

The pharmaceutical preparations may conveniently contain about 1-500 mg, particularly 1-100 mg, of a β secretase inhibitor described herein.

### EXAMPLES

### Example 1—Materials & Methods

Aβ42 administration study: Lyophilised recombinant Aβ₄₂ (Millipore) and scrambled control peptide (ScrAβ₄₂; Millipore) were resuspended in 1% NH₄OH and aliquoted at 200 ng/ml in H₂O and stored at −80° C. for no longer than 4 weeks. Male C57BL6 mice were obtained from the Animal Resource Centre (Perth, Wash.) at 4 weeks of age and housed with 4 mice per cage on a 12 hr light/dark cycle at a temperature of 22° C. and a constant humidity with a normal rodent diet. At 12 weeks of age, mice were grouped according to body mass and composition, determined by EchoMRI. Mice were then administered 1 μg of recombinant Aβ₄₂ or ScrAβ₄₂ (n=10/group per cohort) by i.p. injection once/day for 5 wks. An i.p. glucose tolerance test (GTT) was performed on the final treatment day following an overnight fast. Mice were administered 2 g/kg lean mass of glucose including radioactive glucose tracers, prepared as follows. 100 μl of 1 μCi/μl glucose analogue, [³H]-2-deoxyglucose (2-DOG), and 500 μl of 200 μCi/mL U-¹⁴C glucose were evaporated to dryness before redissolving the radioactive tracers in 1 mL of 50% glucose. This produced a 50% glucose solution containing 100 μCi/mL [³H]-2-DOG and 100 μCi/mL U-¹⁴C glucose. The tail tip of each mouse was cut off and the blood glucose concentration of a blood sample was measured using an AccuCheck II glucometer (Roche). The GTT was initiated via intraperitoneal injection of the radiolabelled glucose solution (2 g/kg body weight, 10 uCi/animal) into the overnight-fasted mice. Further blood samples were taken at 15, 30, 45, 60 and 90 minutes after the injection for the measurement of blood glucose. Blood samples (30 μl) were also taken from the tail tip at each time point and diluted in 100 μl of saline. These samples were then centrifuged and the supernatant collected. 50 μl of the supernatant was diluted in 500 μl of distilled water and then suspended in 4 mL of Ultima Gold XR scintillation fluid (Packard Bioscience). Blood radioactivity was determined at each time point by performing liquid scintillation counting on each solution using the Beckman scintillation counter (LS6000 SC). At the conclusion of the GTT, mice were killed via cervical dislocation. Blood was obtained immediately following by cardiac puncture and the heart, and other tissues were immediately removed. Hearts were washed in ice cold PBS and weighed prior to being snap frozen in liquid nitrogen. The heart (30 mg), epididymal fat pad (30 mg and quadriceps skeletal muscle (30 mg) were homogenised in 1.5 ml of distilled water. The homogenate was centrifuged at 3000 rpm for 10 min at 4° C. 400 μl of the supernatant was diluted into 1.6 mL of distilled water and then suspended in 14 mL of Ultima Gold XR scintillation fluid (Packard Bioscience). The radioactivity of each sample (from both [3H]-2-DOG6P and [³H]-2-DOG) was determined by liquid scintillation counting using the Beckman scintillation counter (LS6000 SC). The ³H radioactivity was used to measure glucose uptake into each tissue.

To determine the incorporation of U-¹⁴C glucose into triglyceride and the total triglyceride content in the heart, an extraction of triglyceride was carried out using a chloroform/methanol mixture. Samples of heart (30 mg) were hand-homogenised in 2 mL of chloroform/methanol (2:1) and the homogeniser rinsed in a further 2 mL of chloroform/methanol (2:1), and the washings being added to the original extract in 10 mL tubes. The tubes were tightly capped and mixed on a rotator overnight to maximise extraction of the triglycerides. 2 ml of 0.6% saline was then added, to facilitate the separation of the organic and aqueous phases, after which the tubes were mixed thoroughly and then centrifuged at 2000 rpm for 10 minutes. The lower chloroform phase (containing triglycerides) was collected and evaporated to dryness under nitrogen at 45° C. The dried extract was then re-dissolved in 250 μl of 100% ethanol, to redissolve the lipid and enable aliquots to dispensed for assay. The amount of U-¹⁴C glucose clearance into the lipid fraction was measured by suspending 100 μl of the triglyceride solution in 5 mL of Ultima Gold XR scintillation fluid (Packard Bioscience), followed by scintillation counting using the Beckman scintillation counter (LS6000 SC). Total triglyceride content was measured using an enzymatic fluorometric assay (BioVision) as per manufacturers' instructions. Lipoprotein lipase was used in an enzymatic reaction to yield fatty acid and glycerol. Quantified glycerol was used as an indirect measure of triglyceride and was normalised to tissue weight.

Total mRNA from the tissues was extracted by homogenizing ˜20-30 milligrams of tissue in 1 ml of Trizol followed by incubation at room temperature (RT) for 5 min. 200 μL of chloroform was added to the homogenate, shaken for 15 seconds and incubated for 1 min at RT before centrifuging at 12,000 g for 10 min at 4° C. for extracting the upper aqueous phase. An equal volume (350 μl for cell lysate/450 μL for tissue) of 70% ethanol was added to cell/tissue samples and they were further purified with RNeasy spin columns (the RNeasy® min i Kit, Qiagen). Complementary DNA (cDNA) was synthesised using the SuperScript™ III transcription system (Invitrogen). cDNA was quantified by OliGreen assay (Quant-iT™ OliGreen® ssDNA Assay Kit; Invitrogen). All primers were designed in-house using the Beacon Primer Designer program software and synthesised by Gene Works (Adelaide, Australia). Primer sequence efficiency was tested over a wide concentration range. Gene expression levels were quantified using the FastStart Universal SYBR Green Master (ROX; Roche Applied-Science) on the MX3005P™ Multiplex Quantitative PCR (QPCR) system (Stratagene). Log-transformed CT values were normalised to cDNA concentration to determine relative gene expression levels.

The effect of Aβ₄₂ administration on cardiac function was assessed in another cohort of 12-week-old, male C57BL6 mice, which were administered Aβ₄₂ or ScrAβ₄₂ (n=10/group per cohort) by i.p. injection once/day for 5 wks. After 4 weeks of peptide administration, cardiac function was assessed by echocardiography as follows. Mice were anaesthetised with inhalation of 1.5% isoflurane anaesthesia and echocardiography was performed using the Phillips HD15 diagnostic ultrasound system with a 15 MHz linear-array transducer by an experienced veterinarian. The velocity of blood flow through the mitral valve was analysed using Doppler mode imaging. These results were used to calculate the deceleration time and E:A ratio. Doppler imaging was also utilised to measure the velocity of blood flow through the aortic valve. The measurements were then used to calculate the ejection time, peak aortic flow and heart rate. M-mode imaging of the left ventricle was used to measure the thickness of the inter-ventricular septum (IVS), left ventricular internal diameter (LVID) and left ventricular posterior wall (LVPW) in both diastole (d) and end-systole (s) as well as systolic measures such as ejection fraction and fractional shortening. An estimation of LV mass was calculated from the m-mode imaging by using the formula (1.05[LVIDd+LVPWd+IVSd]³−[LVIDd]³) by Troy et al. (1972). Mice were humanely killed by cervical dislocation 1 week later. Blood was obtained immediately following by cardiac puncture and the heart, and other tissues were immediately removed. Hearts were washed in ice cold PBS and weighed prior to being snap frozen in liquid nitrogen.

3D6-High Fat Diet (HFD) prevention study: Male C57BL6 mice were obtained from the Animal Resource Centre (Perth, Wash.) at 4 weeks of age and housed 4 mice per cage on a 12 hr light/dark cycle at a temperature of 22° C. and a constant humidity with a normal rodent diet. At 12 weeks of age, echocardiography was performed on all mice (n=24), to obtain pre-treatment measures of cardiac function, as follows. Mice were anaesthetised with inhalation of 1.5% isoflurane anaesthesia and echocardiography was performed using the Phillips HD15 diagnostic ultrasound system with a 15 MHz linear-array transducer by an experienced veterinarian. The velocity of blood flow through the mitral valve was analysed using Doppler mode imaging. These results were used to calculate the deceleration time and E:A ratio. Doppler imaging was also utilised to measure the velocity of blood flow through the aortic valve. The measurements were then used to calculate the ejection time, peak aortic flow and heart rate. M-mode imaging of the left ventricle was used to measure the thickness of the inter-ventricular septum (IVS), left ventricular internal diameter (LVID) and left ventricular posterior wall (LVPW) in both diastole (d) and end-systole (s), as well as systolic measures such as ejection fraction and fractional shortening. An estimation of LV mass was calculated from the m-mode imaging by using the formula (1.05[LVIDd+LVPWd+IVSd]³−[LVIDd]³) by Troy et al. (1972). All mice were then placed on a high fat diet (HFD) with 43% of calories from fat (23.5% by weight; SF04-001 High Fat Rodent Diet Based on D12451, Specialty Feeds, Glen Forrest, Wash.) for 13 weeks. At 12 weeks of age, mice were also administered 0.75 mg/kg bodyweight of either the Aβ₄₂ neutralising antibody 3D6 (#TAB-0809CLV, Creative Biolabs, Shirley, N.Y.) or the InVivo IgG2a Isotype Control antibody (#BE-0085, BioXCell, Lebanon, N.H.) weekly via intraperitoneal (i.p.) injection (n=12/group) for 13 weeks. Groups were selected based on fat mass, body weight and lean mass to match these variables as closely as possible between groups. Each cage contained 2 mice from each group.

After 10 weeks of the treatment period, mice underwent an oral glucose tolerance test (OGTT). Following a 5 hour fast, baseline readings of blood glucose were collected via a tail bleed of the mice using a hand-held glucometer (AccuCheck Performa). Mice were then administered 50 mg of glucose via oral gavage and blood glucose was measured 15, 30, 45, 60- and 90-minutes post administration. An additional 30 μL of blood was collected at baseline and 15, 30- and 60-minutes post administration in heparinised tubes for analysis of serum insulin concentration. Blood was centrifuged at 10,000 g for 10 minutes at 4° C. and plasma was collected by removing the supernatant. Plasma from the OGTT was analysed for insulin content using the Mouse Ultrasensitive Insulin ELISA (ALPCO, Salem, N.H.). An insulin tolerance test (ITT) 11 weeks into the treatment period. Following a 5 hour fast, baseline readings of blood glucose were collected via a tail bleed of mice using a hand-held glucometer (AccuCheck Performa). Mice were administered of humulin via i.p. injection and blood glucose was measured 20, 40, 60, 90- and 120-minutes post administration. Echocardiography was then performed 12 weeks into the treatment period, as described above, to obtain post-treatment measures of cardiac function. Changes in cardiac function parameters were expressed as a percentage of the baseline measure. Mice were sacrificed following 13 weeks of the treatment period. At the conclusion of the treatment period, mice were killed via cervical dislocation following a 5-hr fasting period. Blood was obtained immediately following by cardiac puncture and the heart, and other tissues were immediately removed. Hearts were washed in ice cold PBS and weighed prior to being snap frozen in liquid nitrogen.

3D6-High Fat Diet (HFD) treatment study: At 12 weeks of age, echocardiography was performed on mice (n=36) to obtain baseline measures of cardiac function. Mice were then separated into 3 groups of 12, which included a chow/control, HFD/control and HFD/3D6 group. The groups were selected based on their measures of diastolic function, fat mass and bodyweight, to match these variables as closely as possible. The two HFD groups were then placed on a HFD with 43% of calories from fat (23.5% by weight; SF04-001 High Fat Rodent Diet Based on D12451, Specialty Feeds, Glen Forrest, Wash.) for 22 weeks, while the chow group remained on a standard chow diet. Following 15 weeks of the diet period, echocardiography was again performed on all groups to obtain pre-drug treatment measures of cardiac function. The chow/control and HFD/control groups were then administered 0.75 mg/kg bodyweight of the InVivo IgG2a Isotype Control antibody (#BE-0085, BioXCell, Lebanon, N.H.) weekly via I.P injection for 7 weeks while the HFD/3D6 group received 0.75 mg/kg bodyweight of the 3D6 antibody (#TAB-0809CLV, Creative Biolabs, Shirley, N.Y.). Echocardiography was then performed following 6 weeks of the treatment period to obtain post-drug treatment measures of cardiac function. Following 7 weeks of the drug administration, mice were humanely killed via cervical dislocation and blood was immediately obtained via cardiac puncture and stored in a heparinised tube. The heart, epididymal fat pad, mesenteric fat pad, liver, quadricep, hind limb and brain were then immediately dissected. The heart was blotted prior to being weighed and all tissues were snap frozen in liquid nitrogen and stored at −80° C. Plasma Aβ42 was measured using a high sensitivity ELISA kit (Wako Diagnostics) and plasma that was diluted 1:10 with assay buffer. Cardiac TAG was measured using a triglyceride GPO-PAP kit (Roche Diagnostics) after extraction by KOH hydrolysis.

Aβ₄₀ administration study: Lyophilised recombinant Aβ₄₀ (Millipore) and scrambled control peptide (ScrAβ₄₀; Millipore) were resuspended in 1% NH₄OH and aliquoted at 200 ng/ml in H₂O and stored at −80° C. for no longer than 4 weeks. Male C57BL6 mice were obtained from the Animal Resource Centre (Perth, Wash.) at 4 weeks of age and housed with 4 mice per cage on a 12 hr light/dark cycle at a temperature of 22° C. and a constant humidity with a normal rodent diet. At 12 weeks of age, mice were grouped according to body mass and composition, determined by EchoMRI. Mice were then administered 1 μg of recombinant Aβ₄₀ or ScrAβ₄₀ (n=12/group per cohort) by i.p. injection once/day for 5 wks. After 4 weeks of peptide administration, cardiac function was assessed by echocardiography as follows. Mice were anaesthetised with inhalation of 1.5% isoflurane anaesthesia and echocardiography was performed using the Phillips HD15 diagnostic ultrasound system with a 15 MHz linear-array transducer by an experienced veterinarian. The velocity of blood flow through the mitral valve was analysed using Doppler mode imaging. These results were used to calculate the deceleration time and E:A ratio. Doppler imaging was also utilised to measure the velocity of blood flow through the aortic valve. The measurements were then used to calculate the ejection time, peak aortic flow and heart rate. M-mode imaging of the left ventricle was used to measure the thickness of the inter-ventricular septum (IVS), left ventricular internal diameter (LVID) and left ventricular posterior wall (LVPW) in both diastole (d) and end-systole (s) as well as systolic measures such as ejection fraction and fractional shortening. An estimation of LV mass was calculated from the m-mode imaging by using the formula (1.05[LVIDd+LVPWd+IVSd]³−[LVIDd]³) by Troy et al. (1972). Mice were humanely killed by cervical dislocation 1 week later. Blood was obtained immediately following by cardiac puncture and the heart, and other tissues were immediately removed. Hearts were washed in ice cold PBS and weighed prior to being snap frozen in liquid nitrogen. Plasma Aβ40 was measured using a high sensitivity ELISA kit (Wako Diagnostics) and plasma that was diluted 1:10 with assay buffer.

Verubecestat-High fat diet (HFD) treatment study: At 12 weeks of age, mice (n=30) were placed on a HFD with 43% of calories from fat (23.5% by weight; SF04-001 High Fat Rodent Diet Based on D12451, Specialty Feeds, Glen Forrest, Wash.). After 13 weeks of the diet period, echocardiography was performed on all mice to obtain pre-drug treatment measures of cardiac function. Mice were allocated to treatment groups so that measures of cardiac function and morphology were matched as best as possible. Verubecestat (MedChemExpress) was resuspended in 100% DMSO before being diluted in a hydroxypropyl cellulose solution to give a final solution containing 6.25 mg/mL Verubecestat in 10% hydroxypropyl cellulose and 5% DMSO. A vehicle solution containing 10% hydroxypropyl cellulose and 5% DMSO was also made. Aliquots of both drug and vehicle were stored at −80° C. until required. The HFD/Verubecestat group was administered 25 mg/kg of Verubecestat per day by oral gavage, while the HFD/control group received an equivalent volume of vehicle. Echocardiography was then performed following 4 weeks of the treatment period to obtain post-drug treatment measures of cardiac function. Following 5 weeks of the drug administration, mice were humanely killed via cervical dislocation and blood was immediately obtained via cardiac puncture and stored in a heparinised tube. The heart, epididymal fat pad, mesenteric fat pad, liver, quadricep, hind limb and brain were then immediately dissected. The heart was blotted prior to being weighed and all tissues were snap frozen in liquid nitrogen and stored at −80° C. Plasma Aβ species were measured using high sensitivity ELISA kits (Wako Diagnostics) and plasma that was diluted 1:10 with assay buffer.

### Example 2—Chronic Aβ42 Administration Alters Cardiac Metabolism

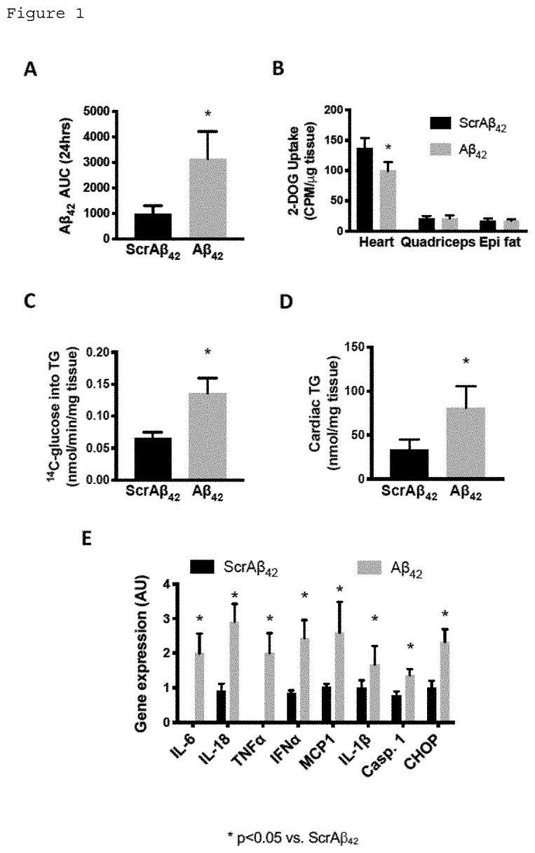

The in vivo effects of Aβ₄₂ were assessed by i.p. administration of 1 μg/day of Aβ₄₂, while control mice were administered a scrambled Aβ₄; peptide (ScrAβ₄₂) for a period of five weeks. Administration of Aβ₄₂ increased plasma Aβ₂ approximately 3-fold compared with administration of ScrAβ₄₂ (FIG. 1A) There was no change in body weight, body composition or food intake in mice administered Aβ₄₂. After five weeks of peptide administration, a GTT with glucose tracers was performed. There was no difference in whole body glucose tolerance or plasma insulin throughout the GTT between ScrAβ₄₂ or Aβ₄₂ administered mice. However, when tissues were assessed for glucose uptake throughout the GTT, by 2-DOG uptake, an ˜25% decrease in glucose uptake by the heart was observed in mice administered Aβ₄₂ (FIG. 1B). Glucose utilisation was further analysed using ¹⁴C-glucose labelling which revealed greater glucose incorporation into TAG (FIG. 1C) and increased total TAG (FIG. 1D) in Aβ₄₂ administered mice. This was associated with gene expression changes indicative of cardiac stress responses, including inflammation and endoplasmic reticulum stress (FIG. 1E).

### Example 3—Chronic Aβ42 Administration Alters Cardiac Function

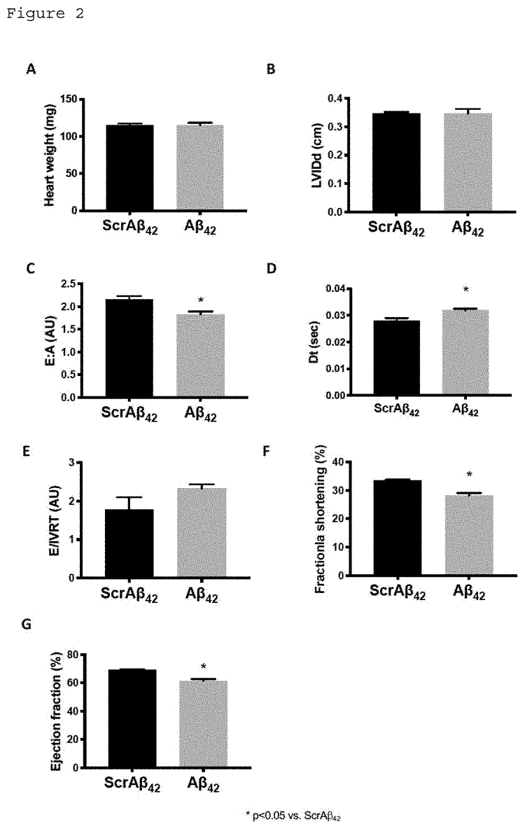

To assess whether Aβ₄₂ administration affected cardiac function, mice were administered ScrAβ₄₂ or Aβ₄₂ for five weeks prior to echocardiography. Hearts were also collected for morphological analysis (FIG. 2). Administration of Aβ₄₂ had no effect on gross heart weight (FIG. 2A) or of internal dimensions of the left ventricle (LVIDd; FIG. 2B). However, indices of diastolic dysfunction were evident in mice administered Aβ₄₂, including reduced E:A ratio (FIG. 2C) and increased deceleration time (FIG. 2D). There was no significant difference between groups when the peak blood flow velocity into the left ventricle during the relaxation phase in early dystole (E) was normalised by the relaxation time (isovolumetric relaxation time; IVRT) (FIG. 2E). This is a index of left atrial pressure and suggests that the diastolic dysfunction observed could be classified as grade 1. Furthermore, fractional shortening (FIG. 2F) and ejection fraction (FIG. 2G) were both reduced in Aβ₄₂ administered mice, which is indicative of systolic dysfunction.

### Example 4—Administration of Anti-Aβ42 Antibodies Preserves Diastolic Function in Development of Obesity

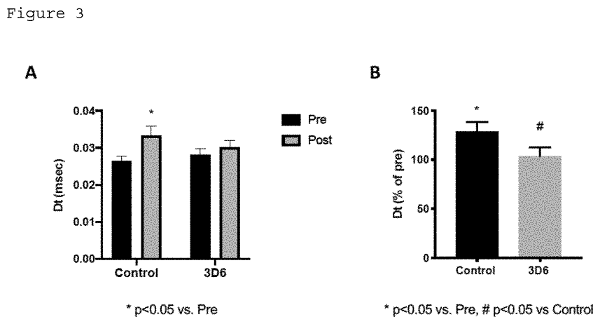

Echocardiography Doppler imaging of the mitral valve was used to assess the deceleration time, a critical measure of diastolic function (FIG. 3). Following 14 weeks of high fat feeding, mice administered the control antibody had an increase in deceleration time (FIG. 3A), indicating deterioration of diastolic function. In contrast, mice administered the 3D6 antibody showed either preserved or decreased deceleration time (FIG. 3A). Expressed relative to baseline measures, mice administered the control antibody had a statistically significant ˜30% increase in deceleration time (FIG. 3B), indicative of diastolic dysfunction. In contrast, deceleration time in mice administered the 3D6 antibody did not change from baseline levels (FIG. 3B). The relative change in deceleration time from baseline was significantly different between control and 3D6 antibody administered groups (FIG. 3B).

### Example 5—Administration of Anti-Aβ42 Antibodies Prevents Concentric Hypertrophy in Development of Obesity

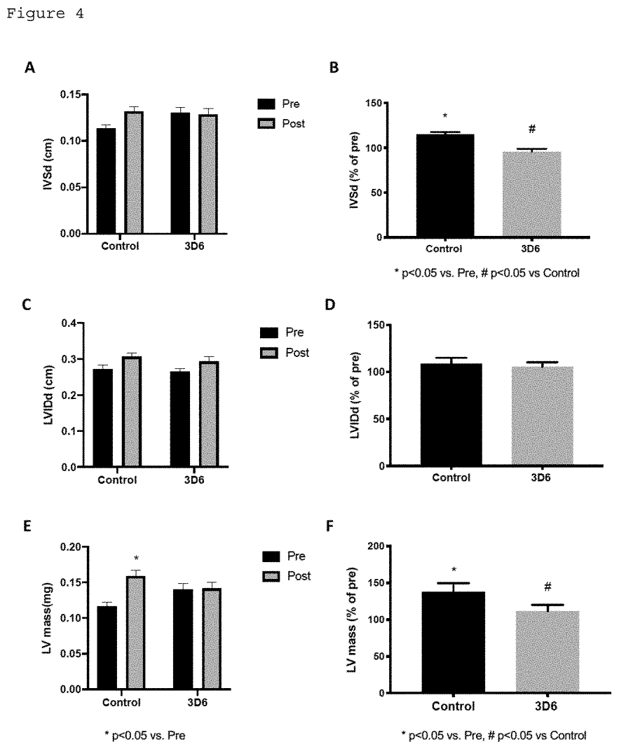

Echocardiographic M-mode imaging was used to characterise the morphology of the left ventricle (FIG. 4). Mice administered control antibody tended to have an increased intraventricular septum thickness at end-diastole (IVSd), a measure of hypertrophy, following the development of obesity, which was not observed in mice administered 3D6 antibody (FIG. 4A). Expressed relative to pre high fat diet values, IVSd significantly increased 115% in mice administered control antibody, while in mice administered 3D6 antibody this value was 95% (FIG. 4B). The relative change in IVSd from pre high fat diet values was significantly different between control and 3D6 antibody administered groups (FIG. 4B). There were no differences between the left ventricle internal diameter at end-diastole (LVIDd), a measure of left ventricle dilation, between groups (FIGS. 4C and D). However, mice administered control antibody significantly increased calculated left ventricular mass, a measure of hypertrophy, throughout the development of obesity, which was not observed in mice administered 3D6 antibody (FIG. 4E). Expressed relative to pre high fat diet values, left ventricular mass significantly increased 138% in mice administered control antibody (FIG. 4F). The relative change in left ventricular mass from pre high fat diet values was significantly different between control and 3D6 antibody administered groups (FIG. 4F).

### Example 6—Administration of Anti-Aβ42 Antibodies Preserves Diastolic Function and Reduces Cardiac TAGs in Established Obesity

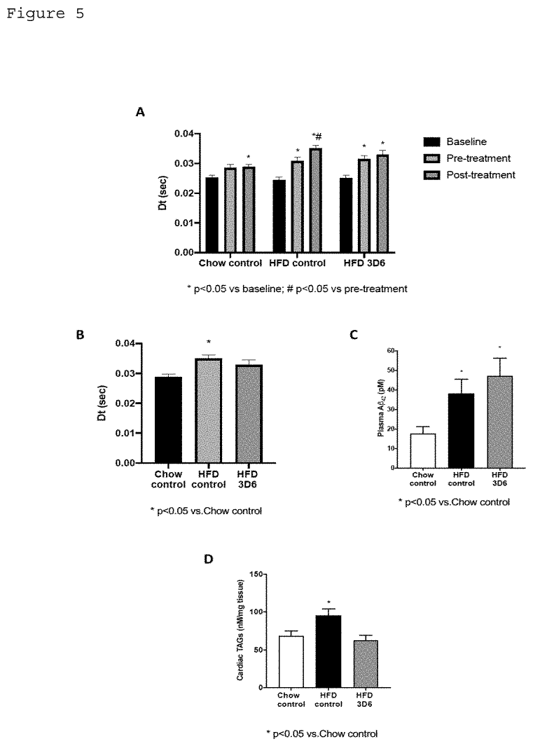

To assess the effect of treating obese mice with 3D6 on diastolic function, Doppler imaging of the mitral valve was conducting using echocardiography at the start of the study (Baseline), after 13 weeks of chow or HFD (Pre-treatment) and following 7 weeks of weekly 3D6 administration (Post-treatment) (FIG. 5). In the chow control group, there was no significant change in DT between Baseline and Pre-treatment, but DT was significantly increased at Post-treatment compared with Baseline (FIG. 5A). In the HFD control group, DT significantly increased from Baseline to Pre-treatment and was further increased at Post-treatment (FIG. 5A). In contrast, the HFD 3D6 group showed a significant increase in DT between Baseline and Pre-treatment, however diastolic function did not deteriorate any further following 3D6 administration (FIG. 5A). When examining DT between groups at the conclusion of treatment period, DT was significantly elevated in the HFD control group compared to the Chow control group, while DT was not significantly different from Chow control in the HFD 3D6 group (FIG. 5B). The effect of the intervention on plasma Aβ42 was examined. In the HFD control group, Aβ42 levels were significantly increased compared with the Chow control group (FIG. 5C). Consistent with the neutralising function of the 3D6 antibody, plasma Aβ42 remained elevated in the HFD 3D6 group compared with Chow control (FIG. 5C). However, 3D6 treatment reduced cardiac TAG accumulation in obese mice (FIG. 5D).

### Example 7—Aβ40 Chronic Administration does not Alter Cardiac Function

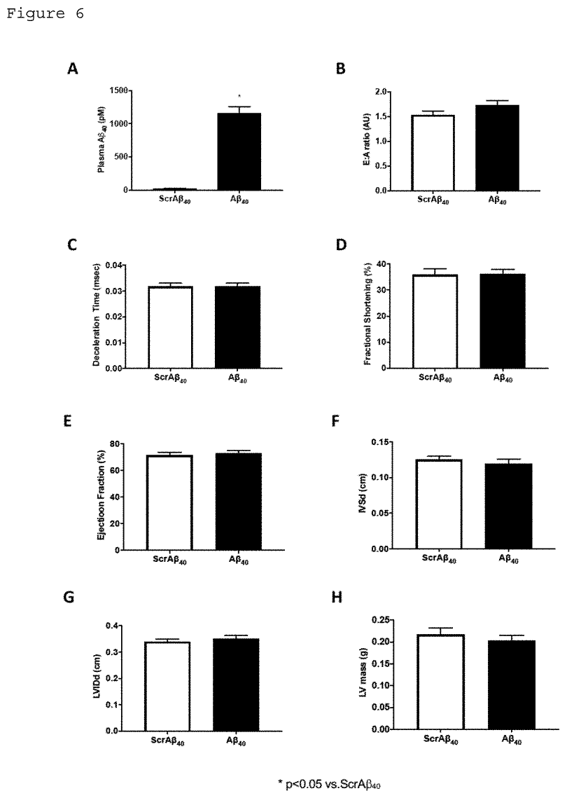

To determine whether other amyloid beta peptides could induce cardiac dysfunction similar to Aβ42, mice were administered Aβ40 or scrambled Aβ40 (ScrAβ40) at 1 μg/day by i.p. injection for 5 weeks, prior to echocardiography (FIG. 6). Administration of Aβ40 significantly increased plasma Aβ40 (FIG. 6A). However, administration of Aβ40 did not have any effect on indices of diastolic function, including E:A ratio (FIG. 6B) and DT (FIG. 6C), nor any effect on indices of systolic function, including fractional shortening (FIG. 6D) and ejection fraction (FIG. 6E). In addition, Aβ40 administration had no effect on cardiac morphology measures, including IVSd (FIG. 6F), LVIDd (FIG. 6G) and LV mass (FIG. 6H).

### Example 8—Administration of β-Secretase Inhibitor Preserves Diastolic Function in Established Obesity

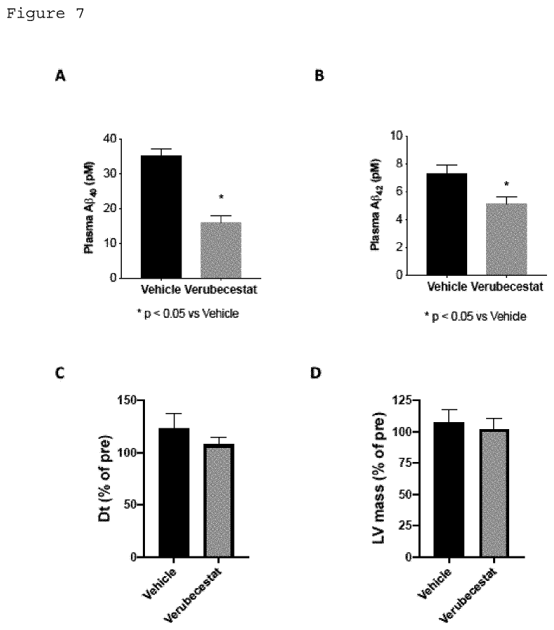

To assess the effect of treating obese mice with the Bace-1 inhibitor Verubecestat on diastolic function, Doppler imaging of the mitral valve was conducting using echocardiography after 13 weeks of HFD (Pre-treatment) and following a further 4 weeks of daily Verubecestat treatment (Post-treatment) (FIG. 7). As a measure of efficacy, Verubecestat decreased both plasma Aβ₄₀ (FIG. 7A) and Aβ₄₂ (FIG. 7B) in obese mice. When examining the % change in DT throughout the treatment period, DT was 122% of baseline levels after treatment in the vehicle group, while DT was 107% of baseline levels after treatment in the Verubecestat group (FIG. 7C). These data suggest that extended treatment with Verubecestat in obese patients or individuals could slow the deterioration in DT in obesity with established cardiac dysfunction. Similar trends were observed for LV mass (FIG. 7D).

### Example 9—Discussion and Conclusion

These data indicate that Aβ42 alters cardiac metabolism and function and has particular impact on diastole. Without being bound by hypothesis, it is believed that the alteration or reprogramming of cardiac metabolism may arise from an Aβ42 mediated or associated inflammatory response.

Administration of Aβ42 to mice reduced cardiac glucose uptake and shunted glucose into TAG synthesis, leading to TAG accumulation. Reduced glucose uptake and utilisation increases the reliance on fatty acid oxidation, which reduces cardiac efficiency. This is due to the greater O₂ cost to produce ATP from beta oxidation, which impairs ATP production and results in impaired cardiac relaxation. This leads to impaired diastolic function because the diastolic relaxation phase has large energetic and ATP requirments, as Ca2+ reuptake and normalisation of membrane ion balances is ATP dependent. Further, the relaxation phase is much longer than systole. Hence the increased reliance on fatty acid oxidation leads to the observed diastolic dysfunction.

Reduced glucose uptake and TAG accumulation are phenotypic traits of cardiomyopathy associated with obesity, whereby altered cardiac metabolism leads to impaired relaxation of the heart, or diastolic dysfunction, which is sufficient to initiate progression to heart failure. Over time, this can lead to concentric hypertrophy and can often also present with impaired systolic function. Consistent with this, administration of Aβ42 to mice impaired both diastolic and systolic function. However these effects on cardiac function were not observed in mice administered Aβ40, suggesting that the effects of amyloid β on the heart are restricted to the isoform of 42 amino acids.

These data also indicate that inhibiting Aβ42 function can prevent the development of diastolic dysfunction in obesity and in other individuals having a higher than normal plasma amount of Aβ42 and protein comprising same. Administration of the 3D6 Aβ42 neutralising antibody to mice throughout high fat feeding prevented the decline in diastolic function and development of concentric hypertrophy, represented by changes in IVSd and left ventricle mass, without left ventricle dilation (LVIDd). These data therefore indicate that β-secretase inhibitors could be used to prevent diastolic dysfunction and progression to heart failure in obesity and conditions in individuals having a higher than normal plasma amount of Aβ42. The data presented support this idea, where Verubecestat administration reduced plasma Aβ₄₂ and influenced the deterioration in deceleration time (DT).

1. A method for preventing or treating diastolic dysfunction in an individual comprising administering to an individual in need of said prevention or treatment a therapeutically effective amount of a β-secretase inhibitor.

2. The method of claim 1 wherein the β-secretase inhibitor is verubecestat.

3. The method of claim 1 wherein the individual has diastolic dysfunction.

4. The method of claim 3 wherein the individual has decreased cardiac glucose uptake.

5. The method of claim 4 wherein the individual has increased glucose incorporation into triacyl glycerol (TAG) in cardiac tissue.

6. The method of claim 5 wherein the individual has increased total cardiac TAG.

7. The method of claim 1 wherein the individual has a reduced E:A ratio.

8. The method of claim 1 wherein the individual has an increased deceleration time.

9. The method of claim 1 wherein the individual has increased intra ventricular septal thickening.

10. The method of claim 1 wherein the individual has increased left ventricle (LV) mass.

11. The method of claim 1 wherein the individual has an elevated plasma amount of Aβ42.

12. The method of claim 1 wherein the β-secretase inhibitor preserves or decreases E wave deceleration, thereby minimising diastolic dysfunction.

13. The method of claim 1 wherein the β-secretase inhibitor prevents concentric hypertrophy.

14. The method of claim 1 wherein the β-secretase inhibitor preserves or prevents intra-ventricular septal thickening.

15. The method of claim 1 wherein the β-secretase inhibitor preserves LV mass or prevents increased LV mass.

16. The method of claim 1 wherein the individual is obese.

17. The method of claim 1 wherein the individual is pre-diabetic, or diabetic.

18. The method of claim 1 wherein the individual does not have Alzheimer's disease.

19. The method of claim 1 wherein the individual has been assessed to determine whether the individual has an elevated amount in plasma of Aβ42.

20. The method of claim 19 wherein the individual is provided with a therapeutically effective amount of a β-secretase inhibitor, where the individual has been assessed as having an elevated plasma amount of Aβ42.
